(19) United States
(12) Patent Application Publication
(10) Pub. No.: US 2024/0180977 A1
(43) Pub. Date: Jun. 6, 2024

(54) COMPOSITIONS AND METHODS RELATING TO CLOSTRIDIUM COCHLEARIUM AND LACTOBACILLUS ACIDOPHILUS

(71) Applicant: Wayne State University, Detroit, MI (US)

(72) Inventor: Kequan Zhou, Detroit, MI (US)

(21) Appl. No.: 18/206,288

(22) Filed: Jun. 6, 2023

Related U.S. Application Data
(60) Provisional application No. 63/349,340, filed on Jun. 6, 2022.

(51) Int. Cl. A61K 35/747; A61K 35/742; A61P 1/14; A61P 3/04; A61P 3/10

(52) U.S. Cl. CPC A61K 35/747; A61K 35/742; A61P 1/14; A61P 3/04; A61P 3/10

(57) ABSTRACT

Methods are provided according to aspects of the present disclosure which improve one or more of: impaired insulin sensitivity, overweight, obesity, glucose homeostasis dysregulation, and gut microbiota profile dysregulation. According to aspects of the present disclosure, methods of improving one or more of: impaired insulin sensitivity, overweight, obesity, glucose homeostasis dysregulation, and gut microbiota profile dysregulation include administering an effective amount of isolated C. cochlearium, isolated L. acidophilus or a combination of isolated C. cochlearium and isolated L. acidophilus, formulated together or separately, in a probiotic composition, to a subject in need thereof.

### REFERENCE TO RELATED APPLICATION

This application claims priority from U.S. Provisional Patent Application Ser. No. 63/349,340, filed Jun. 6, 2022, the entire content of which is incorporated herein by reference.

### GOVERNMENT SUPPORT

This invention was made with government support under Contract No. 1R01AT007566-01A1 awarded by the National Institutes of Health. The government has certain rights in the invention.

### BACKGROUND OF THE INVENTION

Overweight and obesity have been a major public health issue. In 2016, more than 1.9 billion adults were overweight worldwide, of these over 650 million were obese, accounting for 39% and 13% of adults, respectively. Obesity is a primary risk factor for chronic diseases such as type 2 diabetes, cardiovascular diseases, non-alcoholic fatty liver and some cancers. Current approaches to prevent or reduce obesity include lifestyle management involving healthy diets and physical activities, and medical treatment consisting of medicine and surgery.

The human gut is colonized by as many as 10¹⁴ bacterial cells, 10 times more than the number of human body cells. The complex commensal microbiota are essential linkage between the food or nutrients intake and host health. Microbial dysbiosis has been extensively correlated with the development of liver diseases, colorectal cancer, inflammatory bowel disease, as well as obesity and related metabolic syndrome.

Lactobacillus species are Gram-positive, catalase-negative, nonsporulating and facultatively anaerobic organisms, and they are among the most well characterized lactic acid bacteria, see for example, Goldstein E J, et al., Clin Infect Dis 2015; 60 Suppl 2:S98-107.

Clostridium is a large genus of Gram-positive and obligate anaerobic bacteria with great functional diversity, see for example, Yutin N, et al., Environ Microbiol 2013; 15:2631-41. As a butyrate producer, C. cochlearium may be a potential new and safe probiotic against obesity and diabetes. C. cochlearium is also a common commensal bacteria found in the gut of mammalian, such as mice, rabbits and human without association with any harmful effects, see Lee W, et al., (1991) Lab Anim 25:9-15; and Finegold S M, et al., (2002) Clin Infect Dis 35:S6-16.

There is a continuing need for probiotic compositions and methods of their use for providing beneficial effects to a subject. Compositions and methods relating to combinations of C. cochlearium and L. acidophilus (CC-LA) are provided according to aspects of the present disclosure which have beneficial effects, including, but not limited to, beneficial effects on one or more of: body weight control, glucose homeostasis, and gut microbiota profile.

Compositions and methods relating to C. cochlearium and/or butyrates produced by C. cochlearium are provided according to aspects of the present disclosure which have beneficial effects, including, but not limited to, beneficial effects on one or more of: body weight control, and insulin sensitivity.

### SUMMARY OF THE INVENTION

Methods are provided according to aspects of the present disclosure which improve one or more of: impaired insulin sensitivity, overweight, obesity, glucose homeostasis dysregulation, and gut microbiota profile dysregulation. According to aspects of the present disclosure, methods of improving one or more of: impaired insulin sensitivity, overweight, obesity, glucose homeostasis dysregulation, and gut microbiota profile dysregulation include administering an effective amount of isolated C. cochlearium, isolated L. acidophilus or a combination of isolated C. cochlearium and isolated L. acidophilus, formulated together or separately, in a probiotic composition, to a subject in need thereof. According to aspects of the present disclosure, administering the effective amount is via an enteral route. According to aspects of the present disclosure, the subject is human.

According to aspects of the present disclosure, methods of improving one or more of: impaired insulin sensitivity, overweight, obesity, glucose homeostasis dysregulation, and gut microbiota profile dysregulation include administering an effective amount of isolated C. cochlearium, isolated L. acidophilus or a combination of isolated C. cochlearium and isolated L. acidophilus, formulated together or separately, in a probiotic composition, to a subject in need thereof, wherein the effective amount of isolated C. cochlearium, isolated L. acidophilus or a combination of isolated C. cochlearium and isolated L. acidophilus, formulated together or separately, is in the range of 10⁵ CFU to 10¹⁵ CFU, 10⁶ CFU to 10¹⁴ CFU, 10⁷ CFU to 10¹³ CFU, 10⁸ CFU to 10¹² CFU, 10⁹ CFU to 10¹¹ CFU, or 10¹⁰ CFU. According to aspects of the present disclosure, administering the effective amount is via an enteral route. According to aspects of the present disclosure, the subject is human.

According to aspects of the present disclosure, methods of improving one or more of: impaired insulin sensitivity, overweight, obesity, glucose homeostasis dysregulation, and gut microbiota profile dysregulation include administering an effective amount of isolated C. cochlearium, isolated L. acidophilus or a combination of isolated C. cochlearium and isolated L. acidophilus, formulated together or separately, in a probiotic composition, to a subject in need thereof, wherein administering the effective amount comprises a single administration of isolated C. cochlearium, isolated L. acidophilus or a combination of isolated C. cochlearium and isolated L. acidophilus. According to aspects of the present disclosure, administering the effective amount is via an enteral route. According to aspects of the present disclosure, the subject is human.

According to aspects of the present disclosure, methods of improving one or more of: impaired insulin sensitivity, overweight, obesity, glucose homeostasis dysregulation, and gut microbiota profile dysregulation include administering an effective amount of isolated C. cochlearium, isolated L. acidophilus or a combination of isolated C. cochlearium and isolated L. acidophilus, formulated together or separately, in a probiotic composition, to a subject in need thereof, wherein administering the effective amount comprises a multiple administrations of isolated C. cochlearium, isolated L. acidophilus or a combination of isolated C. cochlearium and isolated L. acidophilus. According to aspects of the present disclosure, the multiple administrations are performed at regular intervals selected from daily, weekly, monthly, or more frequently, or less frequently. According to aspects of the present disclosure, administering the effective amount is via an enteral route. According to aspects of the present disclosure, the subject is human.

According to aspects of the present disclosure, the effective amount of isolated C. cochlearium is in the range of 10⁵ CFU/day to 10¹⁵ CFU/day, 10⁶ CFU/day to 10¹⁴ CFU/day, 10⁷ CFU/day to 10¹³ CFU/day, 10¹³ CFU/day to 10¹² CFU/day, 10⁹ CFU/day to 10¹¹ CFU/day, or 10¹⁰ CFU/day.

According to aspects of the present disclosure, the effective amount of isolated L. acidophilus is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

According to aspects of the present disclosure, the effective amount of isolated C. cochlearium is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day, in a combination treatment with isolated L. acidophilus in doses of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

Probiotic compositions according to aspects of the present disclosure include: isolated C. cochlearium, isolated L. acidophilus or a combination of isolated C. cochlearium and isolated L. acidophilus, for improving one or more of: impaired insulin sensitivity, overweight, obesity, glucose homeostasis dysregulation, and gut microbiota profile dysregulation. Probiotic compositions according to aspects of the present disclosure further comprising a pharmaceutically acceptable carrier. Probiotic compositions according to aspects of the present disclosure are formulated as a unit dosage comprising an effective amount of isolated C. cochlearium, isolated L. acidophilus or a combination of isolated C. cochlearium and isolated L. acidophilus. According to aspects of the present disclosure, the unit dosage is in the range of 10⁵ CFU to 10¹⁵ CFU, 10⁶ CFU to 10¹⁴ CFU, 10⁷ CFU to 10¹³ CFU, 10⁸ CFU to 10¹² CFU, 10⁹ CFU to 10¹¹ CFU, or 10¹⁰ CFU.

Probiotic compositions for use in improving one or more of: impaired insulin sensitivity, overweight, obesity, glucose homeostasis dysregulation, and gut microbiota profile dysregulation, according to aspects of the present disclosure include: an effective amount of isolated C. cochlearium, isolated L. acidophilus or a combination of isolated C. cochlearium and isolated L. acidophilus, formulated together or separately. According to aspects of the present disclosure, the effective amount of isolated C. cochlearium, isolated L. acidophilus or a combination of isolated C. cochlearium and isolated L. acidophilus, formulated together or separately, is in the range of 10⁵ CFU to 10¹⁵ CFU, 10⁶ CFU to 10¹⁴ CFU, 10⁷ CFU to 10¹³ CFU, 10⁸ CFU to 10¹² CFU, 10⁹ CFU to 10¹¹ CFU, or 10¹⁰ CFU. According to aspects of the present disclosure, the effective amount of isolated C. cochlearium is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day. According to aspects of the present disclosure, the effective amount of isolated L. acidophilus is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day. According to aspects of the present disclosure, the effective amount of isolated C. cochlearium is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day, in a combination treatment with isolated L. acidophilus in doses of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

Probiotic compositions for use in improving one or more of: impaired insulin sensitivity, overweight, obesity, glucose homeostasis dysregulation, and gut microbiota profile dysregulation, according to aspects of the present disclosure further include a pharmaceutically acceptable carrier.

Probiotic compositions for use in improving one or more of: impaired insulin sensitivity, overweight, obesity, glucose homeostasis dysregulation, and gut microbiota profile dysregulation, according to aspects of the present disclosure are formulated as a unit dosage comprising an effective amount of isolated C. cochlearium, isolated L. acidophilus or a combination of isolated C. cochlearium and isolated L. acidophilus. According to aspects of the present disclosure, the unit dosage is in the range of 10⁵ CFU to 10¹⁵ CFU, 10⁶ CFU to 10¹⁴ CFU, 10⁷ CFU to 10¹³ CFU, 10⁸ CFU to 10¹² CFU, 10⁹ CFU to 10¹¹ CFU, or 10¹⁰ CFU.

### BRIEF DESCRIPTION OF THE DRAWINGS

FIG. 1. Body weight (BW) gains of 17 weeks. Values are means±SEM, n=12, and *P<0.05, **P<0.01, ***P<0.001, and ****P<0.0001 indicating significance between CC-LA and HF groups.

FIG. 2(A) is a graph showing fasting blood glucose at weeks 5, 8, 12 and 16; FIG. 2(B) is a graph showing incremental blood glucose response curves; and FIG. 2(C) is a graph showing incremental areas under curves (AUC) of OGTT at week 17. Values are means±SEM, n=12, and *P<0.05, **P<0.01, ***P<0.001, and ****P<0.0001. FIGS. 2(A) and 2(B) indicating significance between CC-LA and HF groups.

FIG. 3. The relative phylum abundance of gut microbiota between CC-LA and HF groups. n=12, and *P<0.05, **P<0.01, ***P<0.001, and ****P<0.0001.

FIG. 4 shows a set of boxplots of α-diversity indices between CC-LA and HF groups. n=12, and *P<0.05, **P<0.01, ***P<0.001, and ****P<0.0001.

FIGS. 5(A), (B), and (C) are graphs showing: FIG. 5(A) Unweighted; and FIG. 5(B) weighted UniFrac-based PCoA plots with PERMANOVA test, and FIG. 5(C) Bray-Curtis-based NMDS plot with ANOSIM test.

FIGS. 6A, and 6B, show differentially abundant taxa of CC-LA and HF groups identified by LEfSe analysis. FIG. 6 (A) Taxonomic cladogram. Circle diameter is proportional to the taxon abundance. FIG. 6 (B) Histogram plot with LDA scores higher that 2.0. Positive scores represent higher bacterial abundance in HF control, and negative scores represent higher bacterial abundance after the CC-LA intervention. p_, phylum; c_, class; o_, order; f_, family; g_, genus; uni, unidentified.

FIG. 7 shows Spearman correlations between gut microbial species and mouse phenotype variables of CC-LA and HF groups. p_, phylum; c_, class; o_, order; f_, family; g_, genus; s_, species; uni, unidentified.

FIG. 8 is a graph showing body weight (BW) gains of 17 weeks. Values are means SEM, n=12 mice per group. *P<0.05, **P<0.01, ***P<0.001, and ****P<0.0001 indicating significance between CC and HF groups. CC, the group treated with C. cochlearium and fed with high-fat diet; HF, high-fat diet control group; LF, low-fat diet control group.

FIG. 9 is a graph showing fasting blood glucose at weeks 5, 8, 12 and 16. Values are means±SEM, n=12 mice per group. *P<0.05 and **P<0.01 indicating significance between CC and HF groups. CC, the group treated with C. cochlearium and fed with high-fat diet; HF, high-fat diet control group; LF, low-fat diet control group.

FIGS. 10A, 10B, and 10C show results of chemical characterization for the fermentation of C. cochlearium and E. coli in PYG medium. FIG. 10A shows enlarged proton NMR spectra with chemical shift from 5.6-8.7 ppm. FIG. 10B shows proton NMR spectra with chemical shift from 0.0-5.4 ppm. 0.5 mM TSP (sodium salt of 3-trimethylsilylpropionic acid) was used as reference to normalize the concentrations of different samples. FIG. 10C is a graph showing GC-MS measured concentrations of acetate, propionate and butyrate. The vertical bars represent SEM (n=3) for each data point, and different letters represent significant differences (P<0.05). CC refers to C. cochlearium, and EC refers to E. coli.

FIG. 11 is a graph showing gut acetate, propionate, and butyrate in CC, HF and LF groups. The vertical bars represent SEM (n=3 replicates per group) for each data point, and different letters represent significant differences (P<0.05). CC, the group treated with C. cochlearium and fed with high-fat diet; HF, high-fat diet control group; LF, low-fat diet control group.

FIGS. 12A-F are graphs showing results of multivariate analysis of serum metabolites between CC and HF groups (N=19). Principal component analysis (PCA) scores plots (FIGS. 12A and 12D), orthogonal partial least squares-discriminant analysis (OPLS-DA) scores plots (12B and 12E), and permutation validation of OPLS-DA models (12C and 12F) for data collected in positive and negative ionization modes. Data were normalized by Pareto scaling. An ellipse represents the 95% confidence interval using Hotelling's T² statistics. CC, the group treated with C. cochlearium and fed with high-fat diet; HF, high-fat diet control group.

FIG. 13 is a heatmap of Spearman correlations between mouse phenotype variables and key serum biomarkers of CC and HF groups. *P<0.05 and **P<0.01 indicating correlation significance. CC, the group treated with C. cochlearium and fed with high-fat diet; HF, high-fat diet control group.

### DETAILED DESCRIPTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, PA: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

The terms “includes,” “comprises,” “including,” “comprising,” “has,” “having,” and grammatical variations thereof, when used in this specification, are not intended to be limiting, and specify the presence of stated features, elements, and/or components, but do not preclude the presence or addition of one or more other features, elements, components, and/or groups thereof.

The term “about” as used herein in reference to a number is used herein to include numbers which are greater, or less than, a stated or implied value by 1%, 5%, 10%, or 20%.

Particular combinations of features are recited in the claims and/or disclosed in the specification, and these combinations of features are not intended to limit the disclosure of various aspects. Combinations of such features not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a alone; b alone; c alone, a and b, a, b, and c, b and c, a and c, as well as any combination with multiples of the same element, such as a and a; a, a, and a; a, a, and b; a, a, and c; a, b, and b; a, c, and c; and any other combination or ordering of a, b, and c).

The terms “first,” “second,” and the like are used herein to describe various features or elements, but these features or elements are not intended to be limited by these terms, but are only used to distinguish one feature or element from another feature or element. Thus, a first feature or element could be termed a second feature or element, and vice versa, without departing from the teachings of the present disclosure.

Methods and compositions of the present disclosure can be used for prophylaxis as well as amelioration of signs and/or symptoms of one or more of: insulin sensitivity, body weight control, glucose homeostasis, and gut microbiota profile. The terms “treating” and “treatment” used to refer to improving one or more of: impaired insulin sensitivity, overweight, obesity, glucose homeostasis dysregulation, and gut microbiota profile dysregulation, in a subject and include: preventing, inhibiting or ameliorating one or more of: impaired insulin sensitivity, overweight, obesity, glucose homeostasis dysregulation, and gut microbiota profile dysregulation, in the subject, such as slowing progression of one or more of: impaired insulin sensitivity, overweight, obesity, glucose homeostasis dysregulation, and gut microbiota profile dysregulation, and/or reducing or ameliorating a sign or symptom of one or more of: impaired insulin sensitivity, overweight, obesity, glucose homeostasis dysregulation, and gut microbiota profile dysregulation.

A therapeutically effective amount of isolated C. cochlearium administered as a treatment of the present disclosure is an amount which has a beneficial effect in a subject being treated.

A therapeutically effective amount of isolated L. acidophilus administered as a treatment of the present disclosure is an amount which has a beneficial effect in a subject being treated.

A therapeutically effective amount of isolated C. cochlearium and isolated L. acidophilus administered as a combination treatment of the present disclosure is an amount which has a beneficial effect in a subject being treated.

In subjects having one or more of: impaired insulin sensitivity, overweight, obesity, glucose homeostasis dysregulation, and gut microbiota profile dysregulation or at risk for having one or more of: impaired insulin sensitivity, overweight, obesity, glucose homeostasis dysregulation, and gut microbiota profile dysregulation, such as a condition characterized by or other condition responsive to a composition of the present disclosure, a therapeutically effective amount of a composition of the present disclosure is effective to ameliorate or prevent one or more of: impaired insulin sensitivity, overweight, obesity, glucose homeostasis dysregulation, and gut microbiota profile dysregulation.

Combination treatments can allow for reduced effective dosage and increased therapeutic index of the pharmaceutical composition including isolated C. cochlearium and isolated L. acidophilus.

Methods of treatment of a subject having, or at risk of having one or more of: impaired insulin sensitivity, overweight, obesity, glucose homeostasis dysregulation, and gut microbiota profile dysregulation are provided according to aspects of the present disclosure which include administering isolated C. cochlearium, isolated L. acidophilus, or a combination of isolated C. cochlearium and isolated L. acidophilus as a combination formulation or separately, wherein administration of the combination provides a synergistic effect.

Methods of treating one or more of: impaired insulin sensitivity, overweight, obesity, glucose homeostasis dysregulation, and gut microbiota profile dysregulation in a subject in need thereof are provided according to aspects of the present disclosure which include administering isolated C. cochlearium, isolated L. acidophilus, or a combination of isolated C. cochlearium and isolated L. acidophilus, wherein the combination of the isolated C. cochlearium and the isolated L. acidophilus is administered together in a single pharmaceutical formulation, or in separate pharmaceutical formulations.

Methods of treating one or more of: impaired insulin sensitivity, overweight, obesity, glucose homeostasis dysregulation, and gut microbiota profile dysregulation in a subject in need thereof are provided according to aspects of the present disclosure, wherein the . . . is . . . , and which include administering isolated C. cochlearium, isolated L. acidophilus, or a combination of isolated C. cochlearium and isolated L. acidophilus, wherein the combination of the isolated C. cochlearium and isolated L. acidophilus is administered together as a combination formulation or separately.

Methods of treatment of a subject having, or at risk of having one or more of: impaired insulin sensitivity, overweight, obesity, glucose homeostasis dysregulation, and gut microbiota profile dysregulation are provided according to aspects of the present disclosure which include administering isolated C. cochlearium, isolated L. acidophilus, or a combination of isolated C. cochlearium and isolated L. acidophilus as a combination formulation or separately, wherein administration of the combination provides a synergistic effect.

The dosage of isolated C. cochlearium, isolated L. acidophilus, or a combination of isolated C. cochlearium and isolated L. acidophilus and any optional additional therapeutic agent will vary based on factors such as, but not limited to, the route of administration; the age, health, sex, and weight of the subject to whom the composition is to be administered; the nature and extent of the subject's symptoms, if any, and the effect desired. Dosage may be adjusted depending on whether treatment is to be acute or continuing. One of skill in the art can determine a pharmaceutically effective amount in view of these and other considerations typical in medical practice.

According to aspects of the present disclosure, an effective amount of isolated C. cochlearium, isolated L. acidophilus or a combination of isolated C. cochlearium and isolated L. acidophilus, formulated together or separately, is in the range of 10⁵ CFU to 10¹⁵ CFU, 10⁶ CFU to 10¹⁴ CFU, 10⁷ CFU to 10¹³ CFU, 10⁸ CFU to 10¹² CFU, 10⁹ CFU to 10¹¹ CFU, or 10¹⁰ CFU.

In general it is contemplated that a unit dosage of isolated C. cochlearium and/or isolated L. acidophilus is in the range of about 10⁵ CFU to 10¹⁵ CFU each. A unit dose may be administered once, or multiple times, such as daily, weekly, monthly, or more frequently, or less frequently, to obtain the desired effect.

A pharmaceutical composition including: isolated C. cochlearium and/or isolated L. acidophilus, may be formulated for sustained release to obtain desired results.

In particular aspects of inventive methods, isolated C. cochlearium is administered in doses of 10⁵ CFU/day to 10¹⁵ CFU/day, such as 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, 10¹⁰ CFU/day.

In particular aspects of inventive methods, isolated L. acidophilus is administered in doses of 10⁵ CFU/day to 10¹⁵ CFU/day, such as 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

In particular aspects of inventive methods, isolated C. cochlearium is administered in doses of 10⁵ CFU/day to 10¹⁵ CFU/day, such as 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, 10¹⁰ CFU/day, in a combination treatment with isolated L. acidophilus in doses of 10⁵ CFU/day to 10¹⁵ CFU/day, such as 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

In particular aspects of inventive methods, isolated C. cochlearium and isolated L. acidophilus are administered in a ratio (mole:mole) in the range of 0.1:100 to 100:0.1, such as 0.25:50, 0.5:25, 0.75:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:0.75, 25:0.5 or 50:0.25. According to further particular aspects of inventive methods, isolated C. cochlearium and isolated L. acidophilus are administered in a ratio (mole:mole) in the range of 1:1.25, 0.15:1, 0.31:1, 0.63:1, 1.25:1, 12:1, 128:1, 16:1, 2.5:1, 32:1, 64:1 or 1:2.5.

Methods of the present disclosure include administration of a pharmaceutical composition of the present disclosure by enteral administration including, but not limited to, oral, gastric, duodenal, and rectal, routes of administration.

According to aspects of the present disclosure, one or more correlative biomarkers of therapeutic activity of isolated C. cochlearium, isolated L. acidophilus, or a combination of isolated C. cochlearium and isolated L. acidophilus administered to treat one or more of: impaired insulin sensitivity, overweight, obesity, glucose homeostasis dysregulation, and gut microbiota profile dysregulation in a subject in need thereof are assayed to assess treatment of the one or more of: impaired insulin sensitivity, overweight, obesity, glucose homeostasis dysregulation, and gut microbiota profile dysregulation in the subject.

Biomarkers of therapeutic activity of isolated C. cochlearium, isolated L. acidophilus, or a combination of isolated C. cochlearium and isolated L. acidophilus administered to treat one or more of: impaired insulin sensitivity, overweight, obesity, glucose homeostasis dysregulation, and gut microbiota profile dysregulation in a subject in need thereof include, but are not limited to, those listed in Table 4.

Biomarkers of therapeutic activity of isolated C. cochlearium, isolated L. acidophilus, or a combination of isolated C. cochlearium and isolated L. acidophilus are measured according to standard methodologies.

According to aspects of the present disclosure, assays for effects of isolated C. cochlearium, isolated L. acidophilus, or a combination of isolated C. cochlearium and isolated L. acidophilus, are used to monitor a subject. Thus, for example, a test sample is obtained from the subject before treatment according to a method of the present disclosure and at one or more times during and/or following treatment in order to assess effectiveness of the treatment. In a further example, a test sample is obtained from the subject at various times in order to assess the course or progress of disease or healing.

In particular aspects, one or more additional biomarkers are assayed in a test sample obtained from a subject to aid in monitoring treatment with a pharmaceutical composition of the present disclosure. For example, serum is assayed in a test sample obtained from a subject to aid in monitoring treatment with a pharmaceutical composition of the present disclosure.

Methods of treating one or more of: impaired insulin sensitivity, overweight, obesity, glucose homeostasis dysregulation, and gut microbiota profile dysregulation are provided according to aspects of the present disclosure which include obtaining a first sample containing a biomarker from the subject prior to administering isolated C. cochlearium, isolated L. acidophilus, or a combination of isolated C. cochlearium and isolated L. acidophilus, obtaining a second sample containing a biomarker from the subject after administering isolated C. cochlearium, isolated L. acidophilus, or a combination of isolated C. cochlearium and isolated L. acidophilus, and assaying the first and second samples for one or more biomarkers, thereby monitoring effectiveness of administering the isolated C. cochlearium, isolated L. acidophilus, or a combination of isolated C. cochlearium and isolated L. acidophilus.

Standards and controls suitable for assays are well-known in the art and the standard and/or control used can be any appropriate standard and/or control.

Assays for detecting biomarker nucleic acids, particularly mRNA or cDNA, include, but are not limited to, sequencing; polymerase chain reactions (PCR) such as RT-PCR; dot blot; in situ hybridization; Northern blot; and RNase protection.

Immunoassay methods can be used to assay a biomarker, including, but not limited to, enzyme-linked immunosorbent assay (ELISA), enzyme-linked immunofiltration assay (ELIFA), flow cytometry, immunoblot, immunoprecipitation, immunohistochemistry, immunocytochemistry, luminescent immunoassay (LIA), fluorescent immunoassay (FIA), and radioimmunoassay.

According to aspects of the present disclosure, combination therapies include isolated C. cochlearium and isolated L. acidophilus formulated in the same composition. When using separate formulations, the isolated C. cochlearium and isolated L. acidophilus may be administered at the same time, intermittent times, staggered times, prior to, subsequent to, or combinations thereof, with reference to each other.

Methods of treating one or more of: impaired insulin sensitivity, overweight, obesity, glucose homeostasis dysregulation, and gut microbiota profile dysregulation in a subject in need thereof are provided according to aspects of the present disclosure which include administering a combination of isolated C. cochlearium and isolated L. acidophilus, wherein the isolated C. cochlearium and isolated L. acidophilus are administered sequentially within a period of time selected from: one hour, two hours, four hours, eight hours, twelve hours, twenty-four hours, 2 days, 3 days, 4 days, 5 days, 6 days and 7 days.

According to aspects of the present disclosure, isolated C. cochlearium and isolated L. acidophilus may be administered at the same time, intermittent times, staggered times, prior to, subsequent to, or combinations thereof, with reference to each other.

According to aspects of the present disclosure, the isolated C. cochlearium, isolated L. acidophilus, or a combination of isolated C. cochlearium and isolated L. acidophilus are administered daily, administered twice daily or administered more often in one day.

According to aspects of the present disclosure, the isolated C. cochlearium and the isolated L. acidophilus are administered sequentially within a period of time selected from: one hour, two hours, four hours, eight hours, twelve hours, twenty-four hours, 2 days, 3 days, 4 days, 5 days, 6 days or 1 week in a method of treatment of one or more of: impaired insulin sensitivity, overweight, obesity, glucose homeostasis dysregulation, and gut microbiota profile dysregulation in a subject.

According to aspects of the present disclosure, the isolated C. cochlearium, isolated L. acidophilus, or a combination of isolated C. cochlearium and isolated L. acidophilus is administered weekly, twice weekly, three times in a week, every other day, daily, administered twice daily or administered more often in one day. According to aspects of the present disclosure the isolated C. cochlearium is administered less often or more often than the isolated L. acidophilus, in a treatment of one or more of: impaired insulin sensitivity, overweight, obesity, glucose homeostasis dysregulation, and gut microbiota profile dysregulation in a subject.

According to aspects of the present disclosure, the combination of isolated C. cochlearium and isolated L. acidophilus is administered to achieve a synergistic effect of the combined administration in the subject.

According to aspects of the present disclosure, the isolated C. cochlearium or isolated L. acidophilus are administered during a treatment period which can be from 1 day to 100 days, or longer, such as 1 day to 2 days, 2 day to 3 days, 3 day to 5 days, 5 day to 7 days, 7 days to 14 days, 14 days to 21 days, 21 days to 28 days, 28 days to 35 days, 35 days to 50 days or 50 days to 60 days, or longer, and which may include one or more periods in which no treatment is given.

According to aspects of the present disclosure, the isolated C. cochlearium and the isolated L. acidophilus are administered together or separately at the same time, intermittent times, or staggered times during a treatment period which can be from 1 day to 100 days, or longer, such as 1 day to 2 days, 2 day to 3 days, 3 day to 5 days, 5 day to 7 days, 7 days to 14 days, 14 days to 21 days, 21 days to 28 days, 28 days to 35 days, 35 days to 50 days or 50 days to 60 days, or longer, and which may include one or more periods in which the amount of the isolated C. cochlearium and the isolated L. acidophilus is increased or decreased, or in which no treatment is given.

The isolated C. cochlearium, isolated L. acidophilus, or a combination of isolated C. cochlearium and isolated L. acidophilus, and one or more additional therapeutic agents, are administered according to aspects of the present disclosure.

The term “additional therapeutic agent” is used herein to refer to a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues which is a biologically, physiologically, or pharmacologically active substance (or substances) that acts locally or systemically in a subject.

Additional therapeutic agents included according to aspects of methods and compositions of the present disclosure include, but are not limited to, antibiotics, antivirals, antineoplastic agents, analgesics, antipyretics, antidepressants, antipsychotics, anti-cancer agents, antihistamines, anti-osteoporosis agents, anti-osteonecrosis agents, antiinflammatory agents, anxiolytics, chemotherapeutic agents, diuretics, growth factors, hormones, non-steroidal anti-inflammatory agents, steroids and vasoactive agents.

A subject treated according to methods and using compositions of the present disclosure can be mammalian or non-mammalian. A mammalian subject can be any mammal including, but not limited to, a human; a non-human primate; a rodent such as a mouse, rat, or guinea pig; a domesticated pet such as a cat or dog; a horse, cow, pig, sheep, goat, or rabbit. A non-mammalian subject can be any non-mammal including, but not limited to, a bird such as a duck, goose, chicken, or turkey. Subjects can be either gender and can be any age. In aspects of methods including administration of an inventive pharmaceutical composition to a subject, the subject is human. The terms “subject” and “patient” are used interchangeably herein.

Methods and compositions of the present disclosure can be used for prophylaxis as well as amelioration of signs and/or symptoms of one or more of: insulin sensitivity, body weight control, glucose homeostasis, and gut microbiota profile. The terms “treating” and “treatment” used to refer to improving one or more of: impaired insulin sensitivity, overweight, obesity, glucose homeostasis dysregulation, and gut microbiota profile dysregulation, in a subject and include: preventing, inhibiting or ameliorating one or more of: impaired insulin sensitivity, overweight, obesity, glucose homeostasis dysregulation, and gut microbiota profile dysregulation, in the subject, such as slowing progression of one or more of: impaired insulin sensitivity, overweight, obesity, glucose homeostasis dysregulation, and gut microbiota profile dysregulation, and/or reducing or ameliorating a sign or symptom of one or more of: impaired insulin sensitivity, overweight, obesity, glucose homeostasis dysregulation, and gut microbiota profile dysregulation.

A therapeutically effective amount of isolated C. cochlearium administered as a treatment of the present disclosure is an amount which has a beneficial effect in a subject being treated.

A therapeutically effective amount of isolated L. acidophilus administered as a treatment of the present disclosure is an amount which has a beneficial effect in a subject being treated.

A therapeutically effective amount of isolated C. cochlearium and isolated L. acidophilus administered as a combination treatment of the present disclosure is an amount which has a beneficial effect in a subject being treated.

In subjects having one or more of: impaired insulin sensitivity, overweight, obesity, glucose homeostasis dysregulation, and gut microbiota profile dysregulation or at risk for having one or more of: impaired insulin sensitivity, overweight, obesity, glucose homeostasis dysregulation, and gut microbiota profile dysregulation, such as a condition characterized by or other condition responsive to a composition of the present disclosure, a therapeutically effective amount of a composition of the present disclosure is effective to ameliorate or prevent one or more of: impaired insulin sensitivity, overweight, obesity, glucose homeostasis dysregulation, and gut microbiota profile dysregulation.

Combination treatments can allow for reduced effective dosage and increased therapeutic index of the pharmaceutical composition including isolated C. cochlearium and isolated L. acidophilus.

Methods of treatment of a subject having, or at risk of having one or more of: impaired insulin sensitivity, overweight, obesity, glucose homeostasis dysregulation, and gut microbiota profile dysregulation are provided according to aspects of the present disclosure which include administering isolated C. cochlearium, isolated L. acidophilus, or a combination of isolated C. cochlearium and isolated L. acidophilus as a combination formulation or separately, wherein administration of the combination provides a synergistic effect.

Methods of treating one or more of: impaired insulin sensitivity, overweight, obesity, glucose homeostasis dysregulation, and gut microbiota profile dysregulation in a subject in need thereof are provided according to aspects of the present disclosure which include administering isolated C. cochlearium, isolated L. acidophilus, or a combination of isolated C. cochlearium and isolated L. acidophilus, wherein the combination of the isolated C. cochlearium and the isolated L. acidophilus is administered together in a single pharmaceutical formulation, or in separate pharmaceutical formulations.

Methods of treating one or more of: impaired insulin sensitivity, overweight, obesity, glucose homeostasis dysregulation, and gut microbiota profile dysregulation in a subject in need thereof are provided according to aspects of the present disclosure, wherein the . . . is . . . , and which include administering isolated C. cochlearium, isolated L. acidophilus, or a combination of isolated C. cochlearium and isolated L. acidophilus, wherein the combination of the isolated C. cochlearium and isolated L. acidophilus is administered together as a combination formulation or separately.

Methods of treatment of a subject having, or at risk of having one or more of: impaired insulin sensitivity, overweight, obesity, glucose homeostasis dysregulation, and gut microbiota profile dysregulation are provided according to aspects of the present disclosure which include administering isolated C. cochlearium, isolated L. acidophilus, or a combination of isolated C. cochlearium and isolated L. acidophilus as a combination formulation or separately, wherein administration of the combination provides a synergistic effect.

The dosage of isolated C. cochlearium, isolated L. acidophilus, or a combination of isolated C. cochlearium and isolated L. acidophilus and any optional additional therapeutic agent will vary based on factors such as, but not limited to, the route of administration; the age, health, sex, and weight of the subject to whom the composition is to be administered; the nature and extent of the subject's symptoms, if any, and the effect desired. Dosage may be adjusted depending on whether treatment is to be acute or continuing. One of skill in the art can determine a pharmaceutically effective amount in view of these and other considerations typical in medical practice.

In general it is contemplated that a daily dosage of isolated C. cochlearium and/or isolated L. acidophilus is in the range of about 10⁵ CFU to 10¹⁵ CFU each. A daily dose may be administered as two or more divided doses to obtain the desired effect. A pharmaceutical composition including: isolated C. cochlearium and/or isolated L. acidophilus, may also be formulated for sustained release to obtain desired results.

In particular aspects of inventive methods, isolated C. cochlearium is administered in doses of 10⁵ CFU/day to 10¹⁵ CFU/day, such as 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, 10¹⁰ CFU/day.

In particular aspects of inventive methods, isolated L. acidophilus is administered in doses of 10⁵ CFU/day to 10¹⁵ CFU/day, such as 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

In particular aspects of inventive methods, isolated C. cochlearium is administered in doses of 10⁵ CFU/day to 10¹⁵ CFU/day, such as 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, 10¹⁰ CFU/day, in a combination treatment with isolated L. acidophilus in doses of 10⁵ CFU/day to 10¹⁵ CFU/day, such as 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

In particular aspects of inventive methods, isolated C. cochlearium and isolated L. acidophilus are administered in a ratio (mole:mole) in the range of 0.1:100 to 100:0.1, such as 0.25:50, 0.5:25, 0.75:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:0.75, 25:0.5 or 50:0.25. According to further particular aspects of inventive methods, isolated C. cochlearium and isolated L. acidophilus are administered in a ratio (mole:mole) in the range of 1:1.25, 0.15:1, 0.31:1, 0.63:1, 1.25:1, 12:1, 128:1, 16:1, 2.5:1, 32:1, 64:1 or 1:2.5.

Methods of the present disclosure include administration of a pharmaceutical composition of the present disclosure by enteral administration including, but not limited to, oral, gastric, duodenal, and rectal, routes of administration.

According to aspects of the present disclosure, one or more correlative biomarkers of therapeutic activity of isolated C. cochlearium, isolated L. acidophilus, or a combination of isolated C. cochlearium and isolated L. acidophilus administered to treat one or more of: impaired insulin sensitivity, overweight, obesity, glucose homeostasis dysregulation, and gut microbiota profile dysregulation in a subject in need thereof are assayed to assess treatment of the one or more of: impaired insulin sensitivity, overweight, obesity, glucose homeostasis dysregulation, and gut microbiota profile dysregulation in the subject.

Biomarkers of therapeutic activity of isolated C. cochlearium, isolated L. acidophilus, or a combination of isolated C. cochlearium and isolated L. acidophilus administered to treat one or more of: impaired insulin sensitivity, overweight, obesity, glucose homeostasis dysregulation, and gut microbiota profile dysregulation in a subject in need thereof include, but are not limited to, those listed in Table 4.

Biomarkers of therapeutic activity of isolated C. cochlearium, isolated L. acidophilus, or a combination of isolated C. cochlearium and isolated L. acidophilus are measured according to standard methodologies.

According to aspects of the present disclosure, assays for effects of isolated C. cochlearium, isolated L. acidophilus, or a combination of isolated C. cochlearium and isolated L. acidophilus, are used to monitor a subject. Thus, for example, a test sample is obtained from the subject before treatment according to a method of the present disclosure and at one or more times during and/or following treatment in order to assess effectiveness of the treatment. In a further example, a test sample is obtained from the subject at various times in order to assess the course or progress of disease or healing.

In particular aspects, one or more additional biomarkers are assayed in a test sample obtained from a subject to aid in monitoring treatment with a pharmaceutical composition of the present disclosure. For example, serum is assayed in a test sample obtained from a subject to aid in monitoring treatment with a pharmaceutical composition of the present disclosure.

Methods of treating one or more of: impaired insulin sensitivity, overweight, obesity, glucose homeostasis dysregulation, and gut microbiota profile dysregulation are provided according to aspects of the present disclosure which include obtaining a first sample containing a biomarker from the subject prior to administering isolated C. cochlearium, isolated L. acidophilus, or a combination of isolated C. cochlearium and isolated L. acidophilus, obtaining a second sample containing a biomarker from the subject after administering isolated C. cochlearium, isolated L. acidophilus, or a combination of isolated C. cochlearium and isolated L. acidophilus, and assaying the first and second samples for one or more biomarkers, thereby monitoring effectiveness of administering the isolated C. cochlearium, isolated L. acidophilus, or a combination of isolated C. cochlearium and isolated L. acidophilus.

Standards and controls suitable for assays are well-known in the art and the standard and/or control used can be any appropriate standard and/or control.

Assays for detecting biomarker nucleic acids, particularly mRNA or cDNA, include, but are not limited to, sequencing; polymerase chain reactions (PCR) such as RT-PCR; dot blot; in situ hybridization; Northern blot; and RNase protection.

Immunoassay methods can be used to assay a biomarker, including, but not limited to, enzyme-linked immunosorbent assay (ELISA), enzyme-linked immunofiltration assay (ELIFA), flow cytometry, immunoblot, immunoprecipitation, immunohistochemistry, immunocytochemistry, luminescent immunoassay (LIA), fluorescent immunoassay (FIA), and radioimmunoassay.

According to aspects of the present disclosure, combination therapies include isolated C. cochlearium and isolated L. acidophilus formulated in the same composition. When using separate formulations, the isolated C. cochlearium and isolated L. acidophilus may be administered at the same time, intermittent times, staggered times, prior to, subsequent to, or combinations thereof, with reference to each other.

Methods of treating one or more of: impaired insulin sensitivity, overweight, obesity, glucose homeostasis dysregulation, and gut microbiota profile dysregulation in a subject in need thereof are provided according to aspects of the present disclosure which include administering a combination of isolated C. cochlearium and isolated L. acidophilus, wherein the isolated C. cochlearium and isolated L. acidophilus are administered sequentially within a period of time selected from: one hour, two hours, four hours, eight hours, twelve hours, twenty-four hours, 2 days, 3 days, 4 days, 5 days, 6 days and 7 days.

According to aspects of the present disclosure, isolated C. cochlearium and isolated L. acidophilus may be administered at the same time, intermittent times, staggered times, prior to, subsequent to, or combinations thereof, with reference to each other.

According to aspects of the present disclosure, the isolated C. cochlearium, isolated L. acidophilus, or a combination of isolated C. cochlearium and isolated L. acidophilus are administered daily, administered twice daily or administered more often in one day.

According to aspects of the present disclosure, the isolated C. cochlearium and the isolated L. acidophilus are administered sequentially within a period of time selected from: one hour, two hours, four hours, eight hours, twelve hours, twenty-four hours, 2 days, 3 days, 4 days, 5 days, 6 days or 1 week in a method of treatment of one or more of: impaired insulin sensitivity, overweight, obesity, glucose homeostasis dysregulation, and gut microbiota profile dysregulation in a subject.

According to aspects of the present disclosure, the isolated C. cochlearium, isolated L. acidophilus, or a combination of isolated C. cochlearium and isolated L. acidophilus is administered weekly, twice weekly, three times in a week, every other day, daily, administered twice daily or administered more often in one day. According to aspects of the present disclosure the isolated C. cochlearium is administered less often or more often than the isolated L. acidophilus, in a treatment of one or more of: impaired insulin sensitivity, overweight, obesity, glucose homeostasis dysregulation, and gut microbiota profile dysregulation in a subject.

According to aspects of the present disclosure, the combination of isolated C. cochlearium and isolated L. acidophilus is administered to achieve a synergistic effect of the combined administration in the subject.

According to aspects of the present disclosure, the isolated C. cochlearium or isolated L. acidophilus are administered during a treatment period which can be from 1 day to 100 days, or longer, such as 1 day to 2 days, 2 day to 3 days, 3 day to 5 days, 5 day to 7 days, 7 days to 14 days, 14 days to 21 days, 21 days to 28 days, 28 days to 35 days, 35 days to 50 days or 50 days to 60 days, or longer, and which may include one or more periods in which no treatment is given.

According to aspects of the present disclosure, the isolated C. cochlearium and the isolated L. acidophilus are administered together or separately at the same time, intermittent times, or staggered times during a treatment period which can be from 1 day to 100 days, or longer, such as 1 day to 2 days, 2 day to 3 days, 3 day to 5 days, 5 day to 7 days, 7 days to 14 days, 14 days to 21 days, 21 days to 28 days, 28 days to 35 days, 35 days to 50 days or 50 days to 60 days, or longer, and which may include one or more periods in which the amount of the isolated C. cochlearium and the isolated L. acidophilus is increased or decreased, or in which no treatment is given.

The isolated C. cochlearium, isolated L. acidophilus, or a combination of isolated C. cochlearium and isolated L. acidophilus, and one or more additional therapeutic agents, are administered according to aspects of the present disclosure.

The term “additional therapeutic agent” is used herein to refer to a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues which is a biologically, physiologically, or pharmacologically active substance (or substances) that acts locally or systemically in a subject.

Additional therapeutic agents included according to aspects of methods and compositions of the present disclosure include, but are not limited to, antibiotics, antivirals, antineoplastic agents, analgesics, antipyretics, antidepressants, antipsychotics, anti-cancer agents, antihistamines, anti-osteoporosis agents, anti-osteonecrosis agents, antiinflammatory agents, anxiolytics, chemotherapeutic agents, diuretics, growth factors, hormones, non-steroidal anti-inflammatory agents, steroids and vasoactive agents.

A subject treated according to methods and using compositions of the present disclosure can be mammalian or non-mammalian. A mammalian subject can be any mammal including, but not limited to, a human; a non-human primate; a rodent such as a mouse, rat, or guinea pig; a domesticated pet such as a cat or dog; a horse, cow, pig, sheep, goat, or rabbit. A non-mammalian subject can be any non-mammal including, but not limited to, a bird such as a duck, goose, chicken, or turkey. Subjects can be either gender and can be any age. In aspects of methods including administration of an inventive pharmaceutical composition to a subject, the subject is human. The terms “subject” and “patient” are used interchangeably herein.

Compositions are provided according to aspects of the present disclosure which include: isolated C. cochlearium, isolated L. acidophilus, or a combination of isolated C. cochlearium and isolated L. acidophilus. Optionally, a pharmaceutically acceptable carrier is included.

A pharmaceutical composition of the present disclosure may be in any dosage form suitable for administration to a subject, illustratively including solid, semi-solid and liquid dosage forms such as tablets, capsules, powders, granules, suppositories, pills, solutions, suspensions, and gels. Liposomes and emulsions are well-known types of pharmaceutical formulations that can be used to deliver a pharmaceutical agent, particularly a hydrophobic pharmaceutical agent. Pharmaceutical compositions of the present disclosure generally include a pharmaceutically acceptable carrier such as an excipient, diluent and/or vehicle. Delayed release formulations of compositions and delayed release systems, such as semipermeable matrices of solid hydrophobic polymers can be used.

The term “pharmaceutically acceptable carrier” refers to a carrier which is suitable for use in a subject without undue toxicity or irritation to the subject and which is compatible with other ingredients included in a pharmaceutical composition.

Pharmaceutically acceptable carriers, methods for making pharmaceutical compositions and various dosage forms, as well as modes of administration are well-known in the art, for example as detailed in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, PA: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

A solid dosage form for administration or for suspension in a liquid prior to administration illustratively includes capsules, tablets, powders, and granules. In such solid dosage forms, one or more active agents, is admixed with at least one carrier illustratively including a buffer such as, for example, sodium citrate or an alkali metal phosphate illustratively including sodium phosphates, potassium phosphates and calcium phosphates; a filler such as, for example, starch, lactose, sucrose, glucose, mannitol, and silicic acid; a binder such as, for example, carboxymethylcellulose, alginates, gelatin, polyvinylpyrrolidone, sucrose, and acacia; a humectant such as, for example, glycerol; a disintegrating agent such as, for example, agar-agar, calcium carbonate, plant starches such as potato or tapioca starch, alginic acid, certain complex silicates, and sodium carbonate; a solution retarder such as, for example, paraffin; an absorption accelerator such as, for example, a quaternary ammonium compound; a wetting agent such as, for example, cetyl alcohol, glycerol monostearate, and a glycol; an adsorbent such as, for example, kaolin and bentonite; a lubricant such as, for example, talc, calcium stearate, magnesium stearate, a solid polyethylene glycol or sodium lauryl sulfate; a preservative such as an antibacterial agent and an antifungal agent, including for example, sorbic acid, gentamycin and phenol; and a stabilizer such as, for example, sucrose, EDTA, EGTA, and an antioxidant.

Solid dosage forms optionally include a coating such as an enteric coating. The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied having a thickness such that the entire coating does not dissolve in the gastrointestinal fluids at pH below 3 associated with stomach acids, yet dissolves above pH 3 in the small intestine environment. It is expected that any anionic polymer exhibiting a pH-dependent solubility profile is readily used as an enteric coating in the practice of the present disclosure to achieve delivery of the active agent to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and ease of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof. A particular enteric coating material includes acrylic acid polymers and copolymers described for example U.S. Pat. No. 6,136,345.

The enteric coating optionally contains a plasticizer to prevent the formation of pores and cracks that allow the penetration of the gastric fluids into the solid dosage form. Suitable plasticizers illustratively include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g. hydroxypropylcellulose, acids or bases) to solubilize or disperse the coating material, and to improve coating performance and the coated product.

Liquid dosage forms for oral administration include one or more active agents and a pharmaceutically acceptable carrier formulated as an emulsion, solution, suspension, syrup, or elixir. A liquid dosage form of a composition of the present disclosure may include a colorant, a stabilizer, a wetting agent, an emulsifying agent, a suspending agent, a sweetener, a flavoring, or a perfuming agent.

Suitable surface-active agents useful as a pharmaceutically acceptable carrier or excipient in the pharmaceutical compositions of the present disclosure include non-ionic, cationic and/or anionic surfactants having good emulsifying, dispersing and/or wetting properties. Suitable anionic surfactants include both water-soluble soaps and water-soluble synthetic surface-active agents. Suitable soaps are alkaline or alkaline-earth metal salts, non-substituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, non-substituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphthalene sulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidylcholine, dipalmitoylphosphatidyl-choline and their mixtures.

Suitable non-ionic surfactants useful as pharmaceutically acceptable carriers or excipients in the pharmaceutical compositions of the present disclosure include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants useful as pharmaceutically acceptable carriers or excipients in the pharmaceutical compositions of the present disclosure include quaternary ammonium salts, preferably halides, having 4 hydrocarbon radicals optionally substituted with halo, phenyl, substituted phenyl or hydroxy; for instance quaternary ammonium salts containing as N-substituent at least one C8-C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, New Jersey, 1981), “Tensid-Taschenbuch”, 2nd ed. (Hanser Verlag, Vienna, 1981) and “Encyclopaedia of Surfactants (Chemical Publishing Co., New York, 1981).

Structure-forming, thickening or gel-forming agents may be included into the pharmaceutical compositions and combined preparations of the disclosure. Suitable such agents are in particular highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

In particular aspects, a pharmaceutically acceptable carrier is a particulate carrier such as lipid particles including liposomes, micelles, unilamellar or mulitlamellar vesicles; polymer particles such as hydrogel particles, polyglycolic acid particles or polylactic acid particles; inorganic particles such as calcium phosphate particles such as described in for example U.S. Pat. No. 5,648,097; and inorganic/organic particulate carriers such as described for example in U.S. Pat. No. 6,630,486.

A particulate pharmaceutically acceptable carrier can be selected from among a lipid particle; a polymer particle; an inorganic particle; and an inorganic/organic particle. A mixture of particle types can also be included as a particulate pharmaceutically acceptable carrier.

A particulate carrier is typically formulated such that particles have an average particle size in the range of about 1 nm-10 microns. In particular aspects, a particulate carrier is formulated such that particles have an average particle size in the range of about 1 nm-100 nm.

Detailed information concerning customary ingredients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, PA: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

Commercial packages are provided according to aspects of the present disclosure which include isolated C. cochlearium, isolated L. acidophilus, or a combination of isolated C. cochlearium and isolated L. acidophilus, wherein the C. cochlearium and L. acidophilus are provided together in a single pharmaceutical formulation or in separate pharmaceutical formulations.

Instructions for administering the isolated C. cochlearium, isolated L. acidophilus, or a combination of isolated C. cochlearium and isolated L. acidophilus, in combination or separately, are included according to aspects of the disclosure.

One or more ancillary components is optionally included in commercial packages of the present disclosure, such as a buffer or diluent.

Embodiments of inventive compositions and methods are illustrated in the following examples. These examples are provided for illustrative purposes and are not considered limitations on the scope of inventive compositions and methods.

### EXAMPLES

### Example 1

### Materials and Methods

### Bacterial Preparation

C. cochlearium and L. acidophilus were purchased from ATCC (Manassas, VA). They were cultured under anaerobic conditions according to methods described in detail in Udayappan S, et al., NPJ Biofilms Microbi. 2016; 2:e16009; Duncan S H, et al., Appl Environ Microbiol 2004; 70:5810-7; and Louis P, et al., Environ Microbiol 2010; 12:304-14.

At the end of the exponential phase, bacterial culture was collected and centrifuged to remove media. The bacterial pellet was washed with sterile phosphate-buffered saline, and mixed with 25% glycerol in medium to reach the final concentration of 10¹⁰ CFU/mL. Viability was assessed using the most probable number analysis by dilution to extinction and confirmed by microscopic analysis. Samples were stored at −20° C. and used within one week.

### Animal Experiment

Thirty-six 6-week-old male C57BL/6 mice were purchased from Charles River Laboratories (Wilmington, MA), and housed under 12-hour light/dark cycle, controlled humidity (40%±10%) and constant temperature (24° C.±1° C.). After 1 week of acclimatization, mice were randomly assigned into three groups (n=12, 6 mice per cage). The experimental group (CC-LA) was treated with the mixture of C. cochlearium and L. acidophilus (1:1, v/v, 109 CFU/100 μL in sterile water) by gavage of 200 μL/mouse/day, fed ad libitum with high-fat diet. High-fat diet (HF) control and low-fat diet (LF) control groups were treated with the same dose of sterile water as the CC-LA combination. High-fat diet (D12492M) contained 5.24 kcal per gram with 60% of calories from fat and 20% of calories from carbohydrate, low-fat diet (D12450J) contained 3.85 kcal per gram with 10% from fat and 70% from carbohydrate, both were purchased from Research Diets Inc. (New Brunswick, NJ).

The experimental lasted 17 weeks, food intake and body weight were monitored weekly, fasting blood glucose was measured at weeks 5, 8, 12, 16. At week 12, fecal pellets within 24-hour from each cage were collected, and fecal calories was measured on a Bomb Calorimeter (Parr, Moline, IL). The net calorie absorption was calculated by subtracting fecal calorie defecation (cal/day/mouse) from food calorie intake (cal/day/mouse). At week 17, body composition was measured for fat and lean mass, using EchoMRI-100 analyzer (EchoMRI, Houston, TX). At the end, animals were euthanatized by exposing to CO₂, tissues and intestinal content were collected and quenched immediately using liquid nitrogen, stored at −80° C. until use.

### Oral Glucose Tolerance Test

Oral glucose tolerance test (OGTT) was conducted at week 17. After an 8-hour food deprivation with only water given, the mice were administrated with glucose (10% in sterile water) in the dose of 1 g/kg of body weight by gavage. Blood glucose was measured at t=0, 15, 30, 60, and 120 min with an Accu-check glucometer (Roche, Indianapolis, IN). Incremental areas under the curve (AUC) of glucose response were calculated using standard trapezoid method as described in detail in Zhou A L, et al., J Nutr 2015; 145:222-30.

### Homeostatic Model Assessment of Insulin Resistance

In the last week, the fasting insulin was measured using the ultra-sensitive mouse insulin ELISA kit (Crystal Chem, Doners Grove, IL), and homeostatic model assessment of insulin resistance (HOMA-IR) was calculated as follows: [fasting glucose (mg/dL)×fasting insulin level (μU/mL)]/405 as described in Ippagunta S M, et al., J Nutr 2018; 148:510-7.

### Short-Chain Fatty Acids of Gut Content

Small amount of gut content of each mouse was pooled by group. After homogenization, three aliquots of each group were extracted with 0.005 M NaOH in the ratio of 50 mg/l mL. Then, the extracted short-chain fatty acids (SCFAs) were derivatized with propyl chloroformate/pyridine mixture, and analyzed through gas chromatography-mass spectrometry (GC-MS) as described in detail in Zheng X, et al. Metabolomics 2013; 9:818-27; and Kaspar H. Amino acid analysis in biological fluids by gc-ms [PhD]: University of Regensburg; 2009. In this study, retention times for acetate, propionate and butyrate were 2.119, 3.309 and 4.741 min, and quantitative ions were m/z 61, 75 and 71, respectively.

16S rRNA-Amplicon Sequencing of Gut Microbiota

DNA extraction was performed using the Eppedorf EpMotion liquid handling system and following the Qiagen MagAttract PowerMicrobiome kit (previously MoBio PowerMag Microbiome) protocol. DNA samples were quantified using the Quant-iT PicoGreen dsDNA Assay kit. The V4 hypervariable region of the 16S rRNA-encoding gene was amplified on the Illumina MiSeq platform (San Diego, CA, USA) as described in Kozich J J, et al., Appl Environ Microbiol 2013; 79:5112-20. Sequencing data were processed through QIIME (V 1.9.1). After quality filtering and sample assignment, the sequences were denoised and clustered into operational taxonomical units (OTUs) at a 97% cutoff, and taxonomic classification was assigned using the Greengenes database (Release 13.8), as described in detail in Lawley B, Tannock G W. Analysis of 16s rrna gene amplicon sequences using the qiime software package. In: Seymour G, Cullinan M, Heng N, editors. Oral biology. Methods in molecular biology. New York: Humana Press; 2017, p. 153-63; and Lopez-Garcia A, et al., Front Microbiol 2018; 9:e3010.

### Statistical Analysis

Indices of α-diversity, including Chao1, abundance-based coverage estimator (ACE), observed species, Shannon, Inverse-Simpson and Gini-Simpson, were calculated by R software (V 3.6.3) and plotted by GraphPad Prism (V 7.00). β-diversity was measured by the principal coordinate analysis (PCoA) based on UniFrac (unweighted and weighted) distance, and non-metric multi-dimensional scaling (NMDS) with Bray-Curtis distance in R. Permutational multivariate analysis of variance (PERMANOVA) and analysis of similarities (ANOSIM) were further implemented to evaluate the group separation for PCoA and NMDS, respectively.

The linear discriminant analysis (LDA) effect size (LEfSe) was used to identify high-dimensional biomarker taxa with significantly varied relative abundance between groups. The alpha value for the factorial Kruskal-Wallis test among classes was 0.05, threshold on the logarithmic LDA score for discriminative features was 2.0 as described in detail in Moreira G, et al., J Nutr Biochem 2018; 62:143-54; and Segata N, et al., Genome Biol 2011; 12:R60. The non-parametric Spearman's rank correlations between the filtered 66 species (>0.005% abundance) and mouse phenotype were calculated for the CC-LA and HF groups by R software, and the result was presented in a heat map.

Phenotype variables and SCFAs were presented as means±SEM. Statistical analysis was performed using Student's t test by GraphPad Prism (V 7.00). Results were considered statistically significant at P<0.05. Significance were indicated with * for P<0.05, ** for P<0.01, *** for P<0.001, and **** for P<0.0001.

### Results

### CC-LA Combination Reduced Body Weight Gain and Changed Body Composition

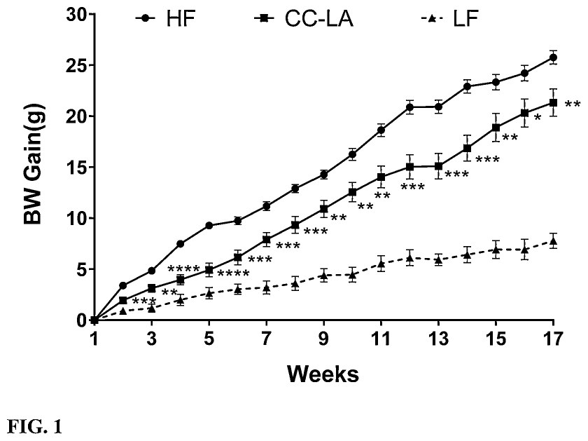

Among the treatment groups, the LF group showed the lowest body weight gain during the dietary treatment. CC-LA group gained significantly less body weight than the HF group did at all weekly check points (P<0.05, FIG. 1). After 17 weeks of dietary intervention, averaged body weight of HF and CC-LA groups were 51.22 g and 45.43 g, respectively (Table 1).

TABLE 1
Body composition, blood glucose and insulin in LF, HF and CC-LA groups at week 17

| | | | | P value | | |
|---|---|---|---|---|---|---|
| | | | | CC-LA | CC-LA | |
| Variable | LF | CC-LA | HF | vs. LF | vs. HF | n |
| BW, g | 30.53 ± 0.80 | 45.43 ± 1.32 | 51.22 ± 0.68 | **** | *** | 12 |
| Fat % | 19.39 ± 2.14 | 41.61 ± 1.82 | 47.10 ± 0.76 | **** | * | 12 |
| Lean % | 66.99 ± 2.13 | 48.17 ± 1.62 | 43.72 ± 0.74 | **** | * | 12 |
| Fat/Lean | 0.30 ± 0.04 | 0.89 ± 0.06 | 1.08 ± 0.03 | **** | ** | 12 |
| Liver, g | 1.12 ± 0.06 | 1.32 ± 0.09 | 1.90 ± 0.10 | 0.0849 | *** | 12 |
| Fasting Glucose, mg/dL | 162.25 ± 13.36 | 191.88 ± 9.44 | 223.75 ± 19.54 | 0.0917 | 0.1639 | 8 |
| Insulin, μU/mL | 57.07 ± 6.35 | 150.71 ± 20.96 | 255.64 ± 36.57 | *** | * | 8 |
| HOMA-IR | 22.39 ± 2.79 | 72.57 ± 11.85 | 143.13 ± 25.90 | *** | * | 8 |

Data are expressed as means ± SEM, and significance are indicated with *P < 0.05, **P < 0.01, ***P < 0.001, and ****P < 0.0001.

Dietary supplementation of CC-LA was able to reduce body weight gain by 4.5 g, a 17.44% reduction compared to HF group (P<0.01, FIG. 1). As shown in Table 1, the CC-LA group also showed significantly lowered fat percentage (P<0.05) but elevated lean percentage than the HF group did (P<0.05). Correspondingly, the CC-LA group had a ratio of fat to lean mass of 0.89 as compared to 1.08 in the HF group (P<0.01). Moreover, liver weights of CC-LA group were significantly lower than that of the HF group (P<0.001).

### CC-LA Combination Improved Blood Glucose Homeostasis

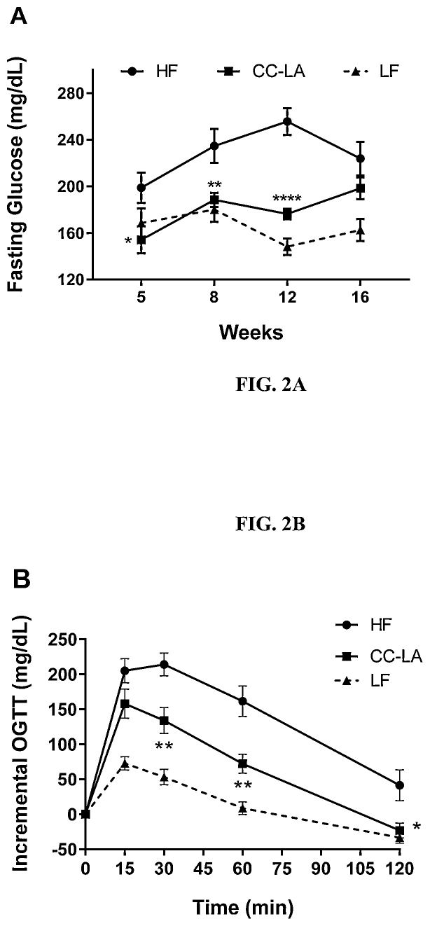

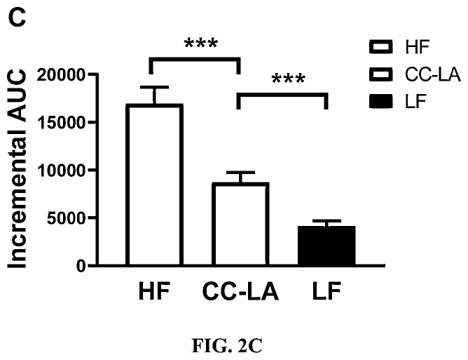

In the fifth week after dietary intervention, the CC-LA group began to show significantly lowered fasting blood glucose than the HF group did (FIG. 2A). The CC-LA group also significantly reduced glucose responses than the HF group did (FIG. 2B). The incremental AUC of the CC-LA group (8691) was 48.6% lower than that of the HF group (16897) (P<0.001, FIG. 2C). In addition, decreased blood insulin and HOMA-IR suggested that the CC-LA combination significantly improved insulin sensitivity (P<0.05, Table 1).

### Effect of CC-LA Combination on Food Intake and Fecal Calories

Food intake and fecal calories were monitored to assess the effect of CC-LA combination on energy homeostasis (Table 2).

TABLE 2
Food intake, fecal calories and gut SCFAs in LF, HF and CC-LA groups

| | | | | P value | | | |
|---|---|---|---|---|---|---|---|
| | | | | CC-LA | CC-LA | LF | |
| Variable | LF | CC-LA | HF | vs. LF | vs. HF | vs. HF | n |
| Food intake | 10385.99 ± 200.20 | 10581.66 ± 305.90 | 11324.80 ± 343.76 | 0.6460 | 0.2477 | 0.1422 | 2 (cages) |
| (cal/day/mouse) | | | | | | | |
| Fecal calories | 788.63 ± 1.94 | 1073.49 ± 6.12 | 1286.90 ± 34.57 | *** | * | ** | 2 (cages) |
| (cal/day/mouse) | | | | | | | |
| Calorie absorption | 9597.3 ± 198.26 | 9508.17 ± 299.78 | 10037.91 ± 309.20 | 0.8272 | 0.3437 | 0.3532 | 2 (cages) |
| (cal/day/mouse) | | | | | | | |
| Acetate | 60.54 ± 0.71 | 67.09 ± 2.17 | 99.37 ± 0.31 | 0.0992 | ** | *** | triplicated |
| (μmol/g) | | | | | | | |
| Propionate | 4.00 ± 0.27 | 4.30 ± 0.20 | 7.38 ± 0.20 | 0.4579 | ** | ** | triplicated |
| (μmol/g) | | | | | | | |
| Butyrate | 5.02 ± 0.10 | 6.59 ± 0.17 | 13.41 ± 0.17 | * | ** | *** | triplicated |
| (μmol/g) | | | | | | | |

Data are expressed as means ± SEM, and significance are indicated with *P < 0.05, **P < 0.01, ***P < 0.001, and ****P < 0.0001.

The averaged calories intake didn't show significant difference between the three groups, including the comparisons between CC-LA and LF, CC-LA and HF, and LF and LF. The CC-LA group had significant lower fecal calories/day/mouse than the HF group, while the LF group showed the lowest fecal calories (P<0.05). As for their net calorie absorption (calorie intake-calorie defecation), no significant difference was observed among all the three groups.

### CC-LA Combination Changed Gut SCFAs

As shown in Table 2, there were no significant difference between the LF and CC-LA groups regarding acetate and propionate. However, the HF group showed significant higher levels of SCFAs than the CC-LA group did.

### CC-LA Combination Modulated Gut Microbial Community

Totally 1,965,862 sequencing reads were obtained from the 24 intestinal content samples, with an average of 81,911 reads per sample (±34,738 SD). 2,144 OTU observations were identified using 97% as a homology cutoff value.

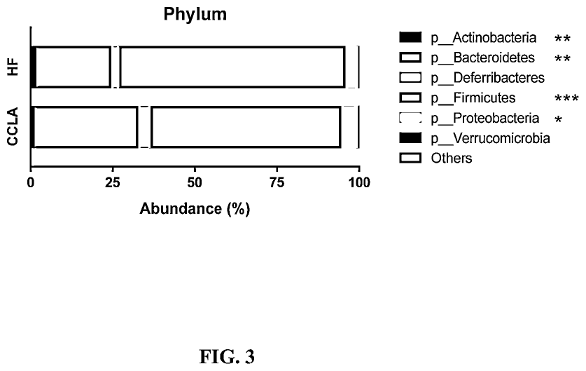

At the phylum level (FIG. 3), Firmicutes, Bacteroidetes and Proteobacteria were the majority, in total accounting for 95.48% and 94.57% of the HF and CC-LA groups, respectively. For the 4 phyla with significant difference (P<0.05, the CC-LA group showed lower relative abundance of Firmicutes (58.03±6.22% vs. 68.88±6.04% in the HF group) and Actinobacteria (0.78±0.40% vs. 1.33±0.46% in the HF group), while higher abundance of Bacteroidetes (31.54±6.29% vs. 22.79±7.10% in the HF group) and Proteobacteria (5.00±1.49% vs. 3.80±1.31% in the HF). The ratio of Firmicutes to Bacteroidetes (F/B) was also reduced from 3.30 in HF group to 1.94 in CC-LA group (P<0.001).

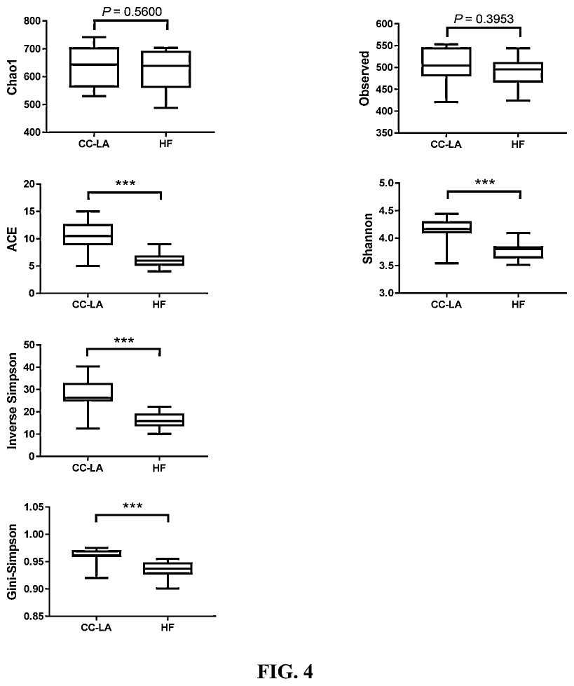

The gut microbiota of the CC-LA group showed higher α-diversity than that of the HF group, in terms of measured indices (FIG. 4). In particular, significant differences were observed for indices ACE, Shannon, Inverse Simpson and Gini-Simpson by t-test (P<0.001).

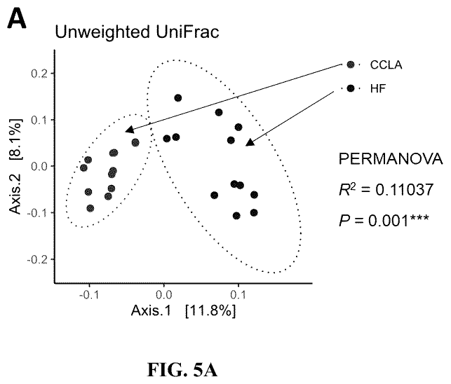

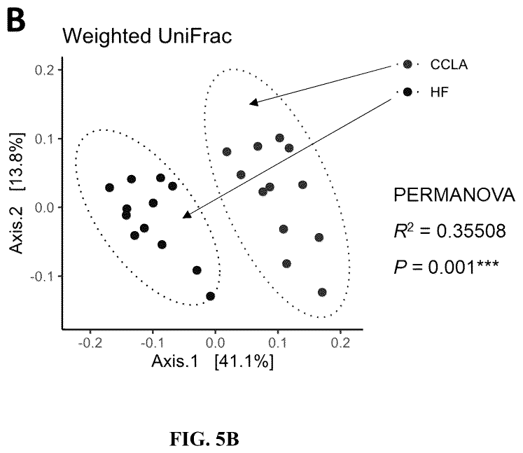

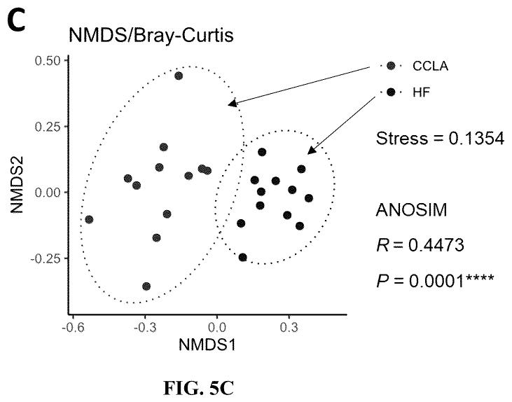

The UniFrac-based PCoA indicated significant separation between the two groups by both weighted (R²=0.35508, P=0.001) and unweighted (R²=0.11037, P=0.001) plots (FIGS. 5A and 5B). The first two components of weighted UniFrac explained 54.9% of the total variance (41.1% and 130.8% for PC1 and PC2), and the first two components of unweighted UniFrac covered 19.9% of the total variance (11.8% and 8.1% for PC1 and PC2). The NMDS plot also suggested that the CC-LA group was significantly distinct from HF group (Stress=0.1354, R=0.4473, P=0.0001), and particularly with respect to the first MDS (FIG. 5C).

### Taxonomic Differences Between the CC-LA and the HF Groups

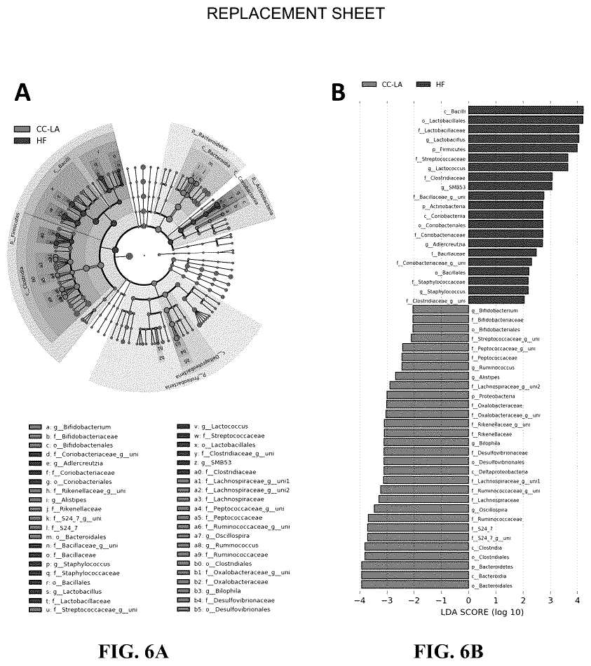

A list of 275 OTUs summarized to genus level was subjected to LEfSe analysis. As shown in the cladogram (FIG. 6A) and LDA score plots (FIG. 6B), the genera of Actinobacteria-Bifidobacterium, Bacteroidetes-Alistipes, Firmicutes-Oscillospira and Ruminococcus, and Proteobacteria-Bilophila were differentially abundant taxa for CC-LA treated group, while the genera of Actinobacteria-Adlercreutzia, Firmicutes-Lactococcus, Lactobacillus, SMB53 and Staphylococcus showed higher relative abundance in HF group.

### Correlation Between Gut Microbiota and Host Phenotype

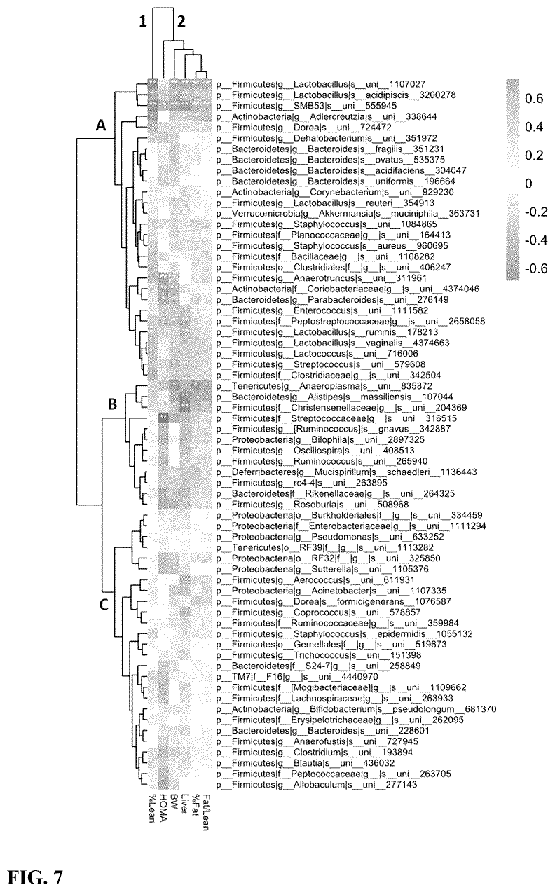

The Spearman correlations between filtered 66 species and mouse phenotype are shown in FIG. 7. The phenotype variables were summed into two main clusters (1 and 2) with contrary features. Correspondingly, the taxon species were divided into three clusters (A, B and C). Cluster A species showed negative correlations with cluster 1 phenotype but positive correlations with cluster 2 phenotype, while cluster B showed an opposite correlation pattern to cluster 1 and 2. In particular, for the species under genera Lactobacillus (OTU 1107027 and 3200278), SMB53 (OTU 555945) and Adlercreutzia (OTU 338644), they showed statistical significance (P<0.05) for both positive correlation with cluster 2, and negative correlation with cluster 1 as demonstrated. The species in cluster C generally showed weak and unclassified correlations with phenotype variables.

Among the cluster 2, HOMA-IR assesses the influence of CC-LA combination on the host insulin resistance. It was distinguished as a sub-cluster by its significant and stronger positive correlations with the species of genera Anaerotruncus (OTU 311961) and Parabacteroides (OTU 276149), and species of families Coriobacteriaceae (OTU 4374046) and Peptostreptococcaceae (OTU 2658058), and negative correlation with the species of family Streptococcaceae (OTU 316515).

This example demonstrates combinations of C. cochlearium and L. acidophilus (CC-LA) have beneficial effects on body weight control and glucose homeostasis in high-fat diet induced obese (DIO) mice. In this study, thirty-six 6-week-old male C57BL/6 mice were randomly assigned to three groups (n=12). The experimental group (CC-LA) was administered with CC-LA mixture and fed ad libitum with high-fat diet. High-fat diet (HF) control and low-fat diet (LF) control groups were treated with the same dose of sterile water as CC-LA group. After 17 weeks of dietary intervention, the CC-LA group showed 17% less body weight gain than the HF group did (P<0.01). The CC-LA group also showed significantly reduced incremental AUC of OGTT and HOMA-IR as compared to the HF group. The results from 16S rRNA sequencing analysis of gut microbiota showed that the CC-LA administration led to overall increased α-diversity indices, and a significant microbial separation from the HF group. The ratio of Firmicutes to Bacteroidetes (F/B) was reduced from 3.30 in the HF group to 1.94 in the CC-LA group. The relative abundances of certain obesity-related taxa were also decreased by CC-LA administration. This example shows that the CC-LA combination reduced obesity and improved glucose metabolism in high fat diet treated DIO mice, potentially mediated by the modulation of gut microbiota.

### Example 2

In this example, production of butyrate and other short chain fatty acids (SCFAs) by C. cochlearium was analyzed using NMR and GC-MS methods. Serum metabolites of mice were profiled by untargeted LC-MS metabolomic analysis, which led to the identification of biomarkers involving the mechanisms of probiotic benefits of C. cochlearium.

Productions of short-chain fatty acids (SCFAs) were characterized for C. cochlearium by NMR and GC-MS analyses. Probiotic effects of C. cochlearium were evaluated through diet induced obese (DIO) C57BL/6 mice. The influence of C. cochlearium administration on gut SCFAs was measured using GC-MS. LC-MS-based untargeted metabolomic profiling and multivariate analysis were used to assess the serum metabolic alteration, identify biomarkers and pathways in response to the C. cochlearium administration.

After 17 weeks of diet intervention, body weight gain of CC group (treated with C. cochlearium and fed with high-fat diet) showed a 21.86% reduction from the high-fat diet (HF) control group (P<0.001), which was specifically reflected on the significantly lowered fat mass (CC vs HF, 17.19 g vs 22.86 g, P<0.0001) and fat percentage (CC vs HF, 41.25% vs 47.10%, P<0.0001), and increased lean percentage (CC vs HF, 46.63% vs 43.72%, P<0.05). C. cochlearium administration significantly reduced fasting blood glucose from week 8 (P<0.05 or 0.01), and eventually improved insulin sensitivity (HOMA-IR, CC vs HF, 63.77 vs 143.13, P<0.05). Overall lowered levels of SCFAs were observed in the gut content of CC group. Metabolomic analysis enabled the identification of 53 discriminatory metabolites and 24 altered pathways between CC and HF groups. In particular, most of the pathway-matched metabolites showed positive correlations with body weight, which included glutamate, phenylalanine, ornithine, PCs, LPCs, AcCas, proline, 5,6-dihydrouracil, pyroglutamic acid, and 1-pyrroline-4-hydroxy-2-carboxylate.

### Methods

Preparation of C. cochlearium

C. cochlearium was purchased from ATCC (Manassas, VA, USA). It was cultured under anaerobic conditions with modified peptone yeast glucose (PYG) medium as described in detail in Udayappan S, et al., NPJ Biofilms Microbi. 2016; 2:e16009; Duncan S H, et al., Appl Environ Microbiol 2004; 70:5810-7; and Louis P, et al., Environ Microbiol 2010; 12:304-14. At the end of the exponential phase, bacterial culture was collected and centrifuged to remove media. The bacterial pellet was washed with sterile phosphate-buffered saline and mixed with 25% glycerol in medium to reach the final concentration of 10¹⁰ CFU/mL. Viability was assessed using the most probable number analysis by dilution to extinction and confirmed by microscopic analysis. Bacteria were stored at −20° C. and used within one week.

### Animal Experiment

Thirty-six 6-week-old male C57BL/6 mice were purchased from Charles River Laboratories (Wilmington, MA, USA). Mice were housed under 12-hour light/dark cycle, controlled humidity (40%±10%) and constant temperature (24° C.±1° C.). After 1 week of acclimatization, mice were randomly assigned into three groups (n=12, 6 mice per cage). The experimental group (CC) was treated with C. cochlearium (10⁹ CFU/100 μL in sterile water) by gavage of 200 μL/mouse/day, fed ad libitum with high-fat diet. High-fat diet (HF) control and low-fat diet (LF) control groups were treated with the same dose of sterile water as the CC group. Mouse diet were purchased from Research Diets Inc. (New Brunswick, NJ, USA). The high-fat diet (D12492M) contained 5.24 kcal per gram with 60% of calories from fat and 20% of calories from carbohydrate, while the low-fat diet (D12450J) contained 3.85 kcal per gram with 10% from fat and 70% from carbohydrate. In addition, the diets included the same amounts of cellulose, minerals, and vitamins.

The experimental lasted 17 weeks, food intake and body weight were monitored weekly, the fasting blood glucose was measured at weeks 5, 8, 12, 16. At week 12, fecal pellets within 24-hour were collected from each cage, and fecal calories was measured on a Bomb Calorimeter (Parr, Moline, IL). The net calorie absorption was calculated by subtracting fecal calorie defecation (cal/day/mouse) from food calorie intake (cal/day/mouse). At week 17, body composition was measured for fat and lean mass using EchoMRI-100 analyzer (Houston, TX, USA). The fasting blood glucose was measured with an Accu-check glucometer (Roche, Indianapolis, IN, USA), and the fasting insulin was measured using the ultra-sensitive mouse insulin ELISA kit (Crystal Chem, Doners Grove, IL, USA). The homeostatic model assessment of insulin resistance (HOMA-IR) was calculated as follows: [fasting glucose (mg/dL)×fasting insulin level (μU/mL)]/405 as described in Ippagunta S M, et al., J Nutr 2018; 148:510-7.

At the end, the blood samples were collected under anesthesia through cardiac puncture, followed with coagulation and centrifugation to obtain the supernatant serum. The serum samples were stored at −80° C. until metabolomic analysis. The mice were finally euthanized by exposing to CO₂. The gut content of cecum and colon was collected and quenched immediately using liquid nitrogen, stored at −80° C. until use.

NMR-Based Characterization of C. cochlearium Fermentation Medium

C. cochlearium and Escherichia coli (from ATCC, Manassas, VA, USA) were both cultured in PYG medium (n=3). Specifically, 0.1 mL (10⁹ CFU) of seeds were inoculated into 10 mL of PYG and incubated under anaerobic condition for C. cochlearium and aerobic conditions for E. coli. Then, their fermented media and a blank PYG were centrifuged to obtain clear supernatant. Samples were further prepared with phosphate buffer (pH 7.4) containing 10% D20 (Cambridge Isotope Laboratories, Inc., Andover, MA, USA), 0.5 mM sodium salt of 3-trimethylsilylpropionic acid (TSP; Sigma-Aldrich, St. Louis, MO, USA) and 1.5 mM NaN₃ (Sigma-Aldrich, St. Louis, MO, USA) as described in detail in Beckonert O, et al. (2007) Nat Protoc 2:2692-2703. The proton NMR spectra were acquired on an Agilent DD2-600 MHz NMR spectrometer (Santa Clara, CA, USA), with 6 in ppm related to TSP and J in Hz. The pulse sequence water_ES was used to suppress water peak and the number of scans was 64.

### GC-MS Measurement of SCFAs in Mouse Gut and Bacterial Media

The concentrations of short-chain fatty acids (SCFAs) in the gut content of mice and culture media of C. cochlearium and E. coli were determined through gas chromatography-mass spectrometry (GC-MS) as described in detail in Kaspar H (2009) Amino acid analysis in biological fluids by GC-MS. PhD thesis, University of Regensburg; and Zheng X, et al. (2013) Metabolomics 9:818-827.

The gut contents were extracted with water containing 5 mM NaOH, in a ratio of 1 mg/50 μL. The culture media were centrifuged to obtain supernatant. Prepared samples were further derivatized by propyl chloroformate (PCF) with a mixture of propanol/pyridine (3:2, v/v), followed by extraction with hexane. The derivatized SCFAs were measured on an Agilent 6890/5973 GC-MS system (Palo Alto, CA, USA) equipped with a Thermo Scientific TG-5MS capillary column (30.0 m×0.25 mm i.d., 0.25 μm film; Waltham, MA, USA). The injection volume was 1 μL. The carrier gas was helium with a flow rate at 2 mL/min. The temperature program was as follows: 40° C. for 2 min, increased to 130° C. at 10° C./min, then increased to 165° C. at 5° C./min, and increased to 300° C. at 80° C./min, held for 2 min. The selected ion monitoring (SIM) mode was applied. The quantitative ions of acetate, propionate and butyrate were m/z 61, 75 and 71, and their retention times were 2.119, 3.309 and 4.741 min, respectively.

### Untargeted LC-MS Metabolomic Analysis of Mouse Serum

Serum samples were thawed on ice, and mixed with chilled HPLC grade methanol (Merck Pvt., Mumbai, IN, USA) in the ratio of 1:3 (v/v) to precipitate protein as described in detail in Dunn W B, et al. (2011) Nat Protoc 6:1060-1083. The mixtures were vortexed for 1 min, followed by centrifugation for 5 min at 17 G. The supernatants were carefully transferred into autosampler vial and stored at 4° C. until use. A quality control (QC) sample was prepared by pooling equal volume of each serum, a blank sample was prepared using water, and both were treated following the same procedure as serum samples.

The metabolomic profiling of serum samples was performed on a Dionex UltiMate 3000 RSLC UHPLC system (Thermo Scientific, Waltham, MA, USA) coupled with a quadrupole-orbitrap mass spectrometer (Q Exactive, HF Hybrid; Thermo Scientific, Waltham, MA, USA), and equipped with an ACQUITY UPLC BEH HILIC column (130 Å, 1.7 μm, 2.1 mm×150 mm; Waters, Milford, MA, USA). The mobile phase consisted of solvents A (10 mM ammonium formate with 0.1% formic acid, v/v) and B (acetonitrile with 0.1% formic acid, v/v). The gradient elution was as follows: 0-0.5 min, 95% of B; 0.5-9.0 min, from 95 to 40% of B; 9.0-9.1 min, from 40 back to 95% of B; 9.1-10.0 min, 95% of B. The flow rate was 0.4 mL/min, the column was kept at 30° C., and the injection volume was 2 μL. The mass spectrometry was operated in full scan mode with a resolution of 240,000 full width at half maximum (FWHM) at 200 m/z. The data were acquired in both positive and negative ionization modes with a mass range of 70-1050 m/z.

Due to inadequate serum amounts for 2 mice in LF group, 2 in HF group and 3 in CC group, ultimately 29 serum samples were chemically profiled in a randomized order started from the fifth injection. The QC samples were analyzed as the first three injections, then every seventh injection. The blank sample was run twice with the first one as the fourth injection, while another one as the injection 26.

### Metabolomic Data Analysis and Biomarker Identification

The software Compound Discoverer 3.0 (CD; Thermo Scientific, Waltham, MA, USA) was used to align, integrate and normalize the chromatographic peaks, and produce metabolic features (m z and retention time) based on the pooled QC sample described in detail in Lu D, et al. (2019) J Chromatogr A 1589:105-115; and Gong J, et al. (2021) JCI Insight 6:e137594. The generated feature list was filtered to remove those peaks presented in less than 50% of QC samples, had areas less than 5 times the background, and with relative standard deviation (RSD) greater than 30% across QC samples. To reduce the statistical influence of greatly different variance of particular metabolites, the filtered data was further normalized using Pareto scaling. Principal component analysis (PCA), and an orthogonal partial least squares-discriminant analysis (OPLS-DA) with permutation test were performed for data acquired in positive and negative ionization modes, respectively, using the SIMCA-P software (Version 14.0.1, Umetrics, Umeaå, Sweden). Thereafter, discriminatory features between HF and CC groups were generated based on the criteria that values of Variable Importance in the Projection (VIP) equal to or more than 2, and two tailed Student's t-test P-value<0.05.

Potential molecular formulas of metabolic features were calculated according to their accurate masses and isotope patterns. The database mzCloud, mzVault and ChemSpider were searched to identify potential candidates, and their hit accuracies were manually confirmed as well. Only metabolites with high matches to the MS/MS libraries were assigned the names of compounds. Identified differential biomarkers were further uploaded to the platform MetaboAnalyst 5.0 (https://www.metaboanalyst.ca/MetaboAnalyst/home.xhtml) for enrichment analysis, as well as pathway searching through the Mus musculus (mouse) library of Kyoto Encyclopedia of Genes and Genomes (KEGG). Spearman's rank correlations between 24 biomarkers hit pathways and 6 phenotype variables were calculated for CC and HF groups using the “psych” package in R (V 4.0.4), and the result was presented in a heat map using the “pheatmap” package.

### Statistical Analysis

Normal distribution was analyzed by Anderson-Darling, D'Agootino-Pearson, Shapiro-Wilk, or Kolmogoov-Smirnov in GraphPad Prism (V 7.00). The results of body weight and composition (n=12 mice per group), fasting blood glucose (n=12 mice per group), insulin resistance (n=8 mice per group), and energy homeostasis (n=2 cages per group) were presented as means±SEM. Statistical comparisons between two groups were analyzed using Student's t test. Significance was indicated with * for P<0.05, ** for P<0.01, *** for P<0.001, and **** for P<0.0001. SCFA concentrations of mouse gut content and bacterial fermentation were also expressed as means±SEM (n=3 replicates per group). Their statistical significance among three groups was compared by one-way ANOVA and Tukey's test (P<0.05), using the SPSS software for Windows (version 25, 2017, IBM).

### Results

C. cochlearium Treatment Improved Body Composition

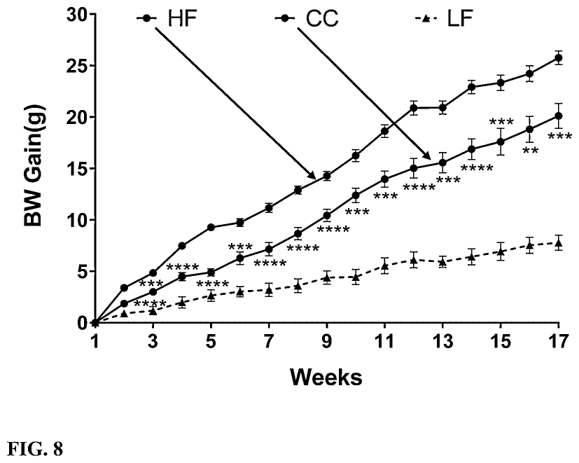

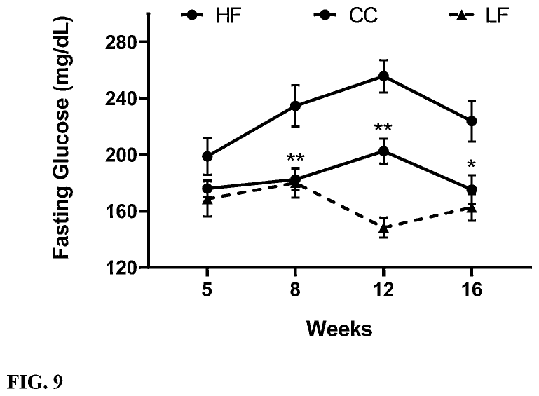

The initial body weights at week 1 were 22.75 g for LF group, 23.59 g for CC groups, and 25.46 g for HF group. As shown in FIG. 8, the LF control group showed the least body weight gain throughout the dietary treatment. The C. cochlearium treatment showed significantly lowered body weight gain than the HF group did from week 2 to the end of the dietary treatment (P<0.01). At the end of the treatment, the averaged body weight gain of HF, CC and LF groups were 25.75 g, 20.12 g and 7.78 g, respectively. The HF and CC groups gained body weights 3.3 and 2.5 times the gain of LF group due to the feeding of high fat-diet. While the C. cochlearium supplementation reduced body weight gain by 5.63 g, a 21.86% reduction compared to HF group (P<0.001, FIG. 8). Eventually, the LF group exhibited the lowest absolute body weight at week 17, while the CC group was 7.55 g lower than HF group, a 14.74% reduction (P<0.0001, Table 3). In terms of body composition, the three groups showed similar lean mass (19-21 g), however, the fat masses of HF and CC (22.86 g and 17.19 g) were more than 3 times of the LF group (5.74 g), see Table 3.

TABLE 3
Body composition, insulin resistance and energy homeostasis of LF, HF and CC groups

| | | | | P value | | | |
|---|---|---|---|---|---|---|---|
| | | | | CC | CC | LF | |
| Variable | LF | CC | HF | vs. LF | vs. HF | vs. HF | n |
| BW at week 17, g | 30.53 ± 0.80 | 43.67 ± 1.20 | 51.22 ± 0.68 | **** | **** | **** | 12 |
| Fat mass, g | 5.74 ± 0.64 | 17.19 ± 0.64 | 22.86 ± 0.57 | **** | **** | **** | 12 |
| Fat % | 19.39 ± 2.14 | 41.25 ± 0.88 | 47.10 ± 0.76 | **** | **** | **** | 12 |
| Lean mass, g | 19.98 ± 0.91 | 19.34 ± 0.44 | 21.19 ± 0.40 | 0.5362 | ** | 0.2407 | 12 |
| Lean % | 66.99 ± 2.13 | 46.63 ± 0.82 | 43.72 ± 0.74 | **** | * | **** | 12 |
| Fat/Lean | 0.30 ± 0.04 | 0.89 ± 0.03 | 1.08 ± 0.03 | **** | *** | **** | 12 |
| Insulin, μU/mL | 57.07 ± 6.35 | 140.01 ± 18.82 | 255.64 ± 36.57 | *** | * | *** | 8 |
| HOMA-IR | 22.39 ± 2.79 | 63.77 ± 12.11 | 143.13 ± 25.90 | ** | * | *** | 8 |
| Food intake, | 10385.99 ± 200.20 | 10660.97 ± 90.93 | 11324.80 ± 343.76 | 0.3376 | 0.2029 | 0.1422 | 2 (cages) |
| cal/day/mouse | | | | | | | |
| Fecal calories, | 788.63 ± 1.94 | 1120.44 ± 18.38 | 1286.90 ± 34.57 | ** | 0.0511 | ** | 2 (cages) |
| cal/day/mouse | | | | | | | |
| Calorie absorption, | 9597.36 ± 198.26 | 9540.52 ± 109.31 | 10037.91 ± 309.20 | 0.8252 | 0.2686 | 0.3532 | 2 (cages) |
| cal/day/mouse | | | | | | | |

Data are expressed as means ± SEM, and significance are indicated with *P < 0.05, **P < 0.01, ***P < 0.001, and ****P < 0.0001. CC refers to the group treated with C. cochlearium and fed with high-fat diet; HF is high-fat diet control group, and LF is low-fat diet control group.

Furthermore, C. cochlearium treatment resulted in significantly lowered fat mass (P<0.0001) and fat percentage (P<0.0001), while increased lean percentage (P<0.05) than the CC group. The ratio of fat to lean mass of CC group was reduced to 0.89 from 1.08 of the HF group (P<0.001).

C. cochlearium Treatment Improved Insulin Sensitivity

At week 5 of the treatment, the CC group began to show a trend of lowered fasting blood glucose than the HF group (FIG. 9). The difference became significant at week 8 until the end of experiment. At week 16, the fasting blood glucose were 223.8 mg/dL for HF group, 175.1 mg/dL for CC, and 162.6 mg/dL for LF. Meanwhile, the CC group had a reduced insulin level of 140.01 μU/mL as compared to the level of 255.64 μU/mL for HF group (P<0.05). Moreover, the CC group had a significantly lowered HOMA-IR (63.77) than the HF group did (P<0.05), suggesting an improved insulin sensitivity by C. cochlearium treatment (Table 3).

Effects of C. cochlearium Treatment on Energy Homeostasis

Effects of C. cochlearium treatment on food intake and fecal calories were included in Table 3. The averaged calories (cal/day/mouse) intake didn't show significant difference between CC and LF, and CC and HF groups. The CC group exhibited significantly higher fecal calories than the LF group (P<0.01), while no significant difference from the HF group. As for their net calorie absorption (calorie intake minus calorie defecation), no significant difference was observed among the three groups.

Chemical Characterization of C. cochlearium Fermentation

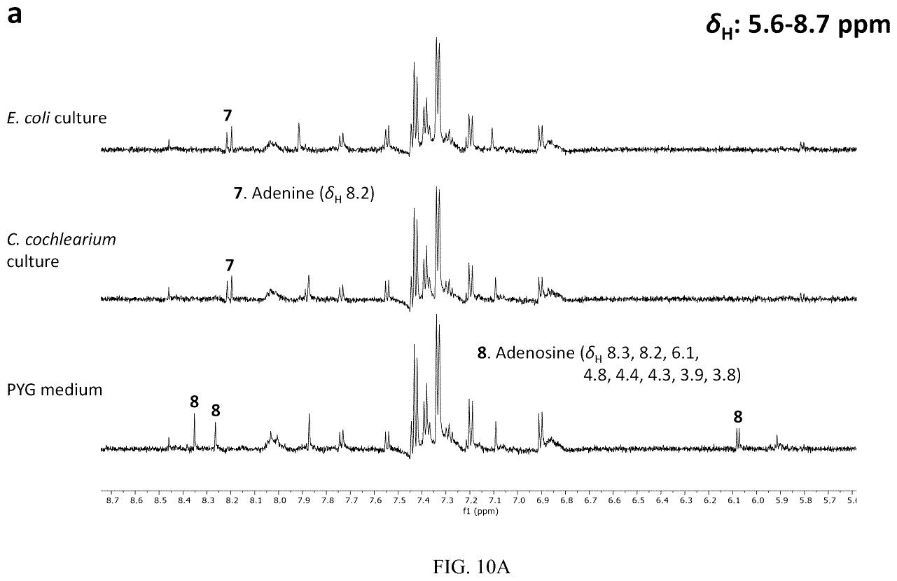

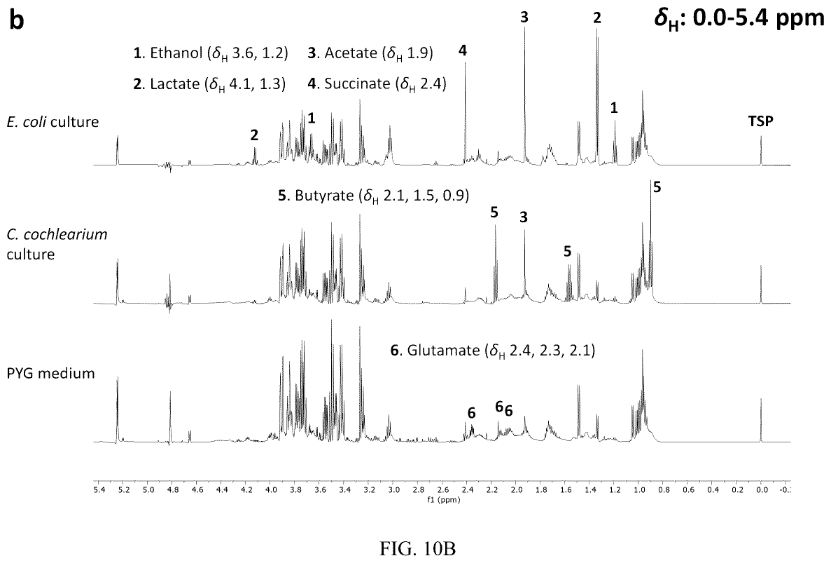

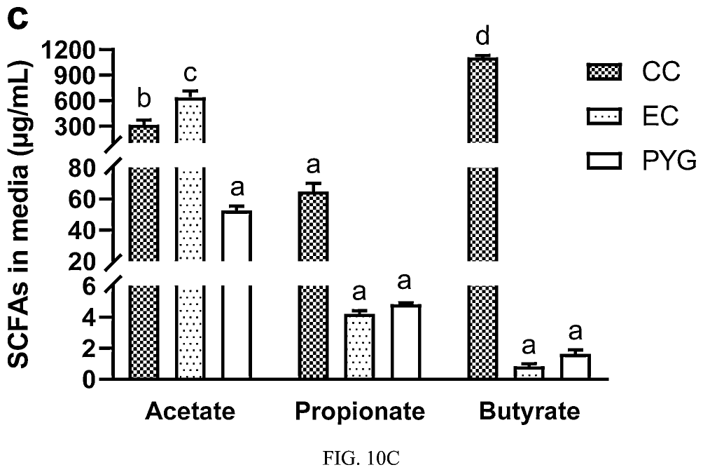

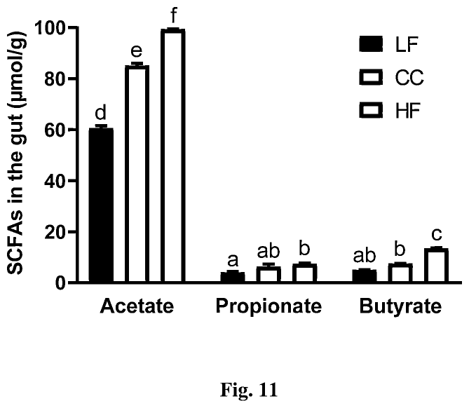

The metabolites of C. cochlearium fermentation were profiled by NMR and then GC-MS methods. E. coli is not a butyrate producer, it was used as a negative control to distinguish the metabolites of C. cochlearium. Water suppressed proton NMR for cultures of C. cochlearium and E. coli in PYG medium. Enlarged and annotated spectra were shown in FIGS. 10A and 10B. Comparing to the PYG control, it was evident that adenosine was consumed, while adenine and acetate were generated by both bacteria. In addition, E. coli produced ethanol, lactate and succinate. It is worth noting that C. cochlearium excreted butyrate and consumed glutamate. FIG. 10C showed the quantitative results of SCFAs in bacterial fermentation by GC-MS. Both bacteria significantly produced acetate (P<0.05), while E. coli (637.27 μg/mL) more than doubled the amount C. cochlearium produced (314.05 μg/mL). There was no significant change for propionate. In particular, C. cochlearium was able to generate butyrate up to 1106.29 μg/mL, while E. coli showed no change for butyrate.

C. cochlearium Treatment Modified SCFAs of Gut Content

As for the three SCFAs in mouse gut content, HF group possessed the highest concentrations among the three groups (FIG. 11, P<0.05). CC group had the higher level of acetate than LF, but no significant difference with LF on propionate and butyrate.

C. cochlearium Treatment Altered Serum Metabolites and Pathways

LC-MS-based untargeted method was used to profile the serum metabolomic modification caused by C. cochlearium supplementation. A list of 5,825 feature metabolites, including 3,492 positive and 2,333 negative features, was generated from the raw data processing and filtration.

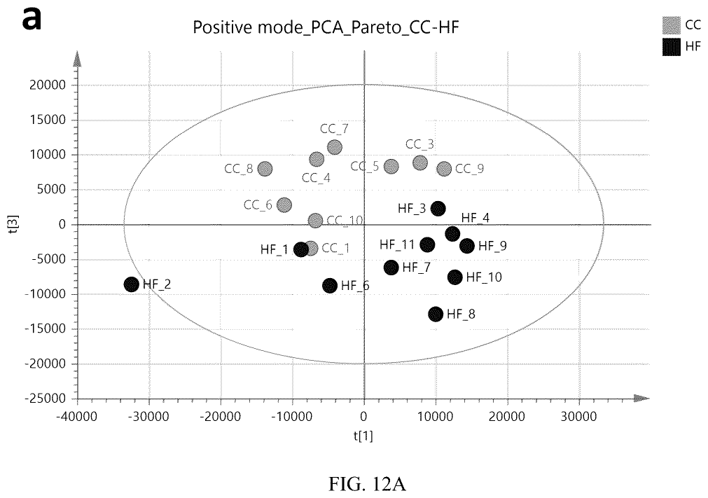

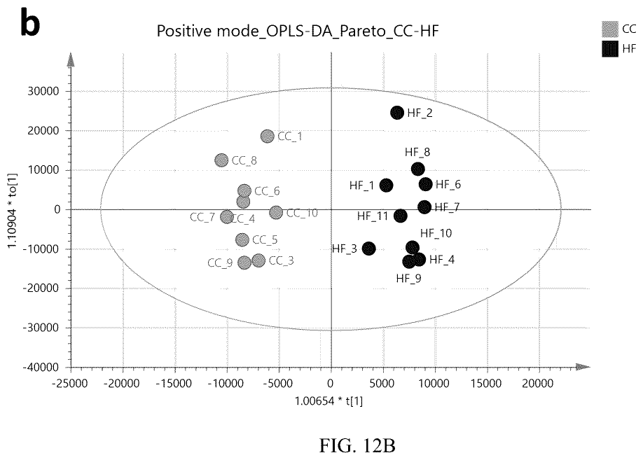

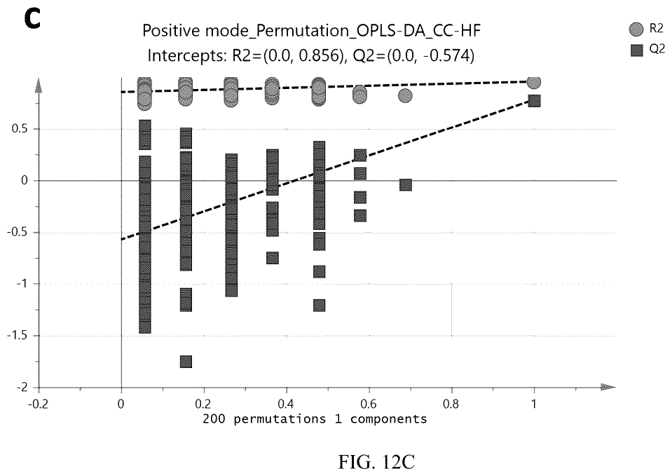

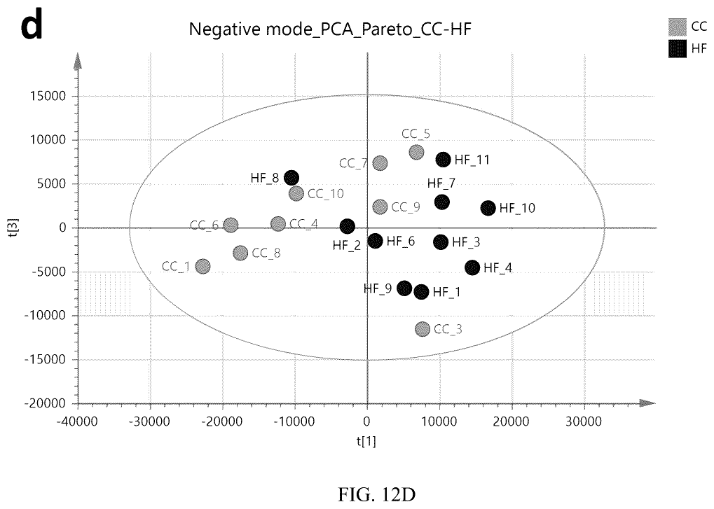

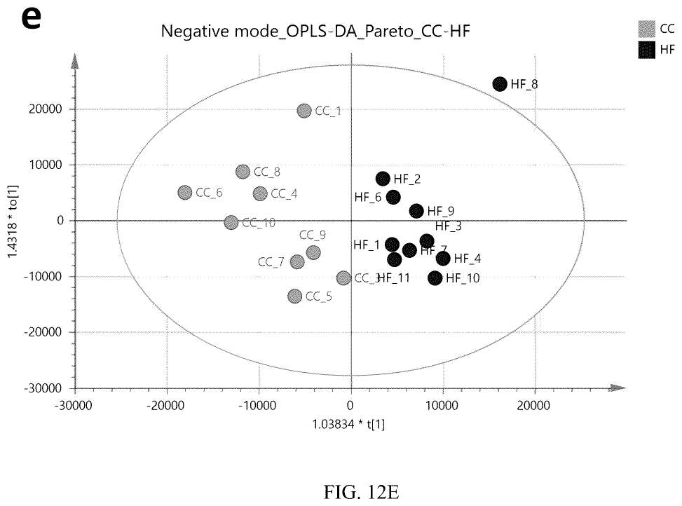

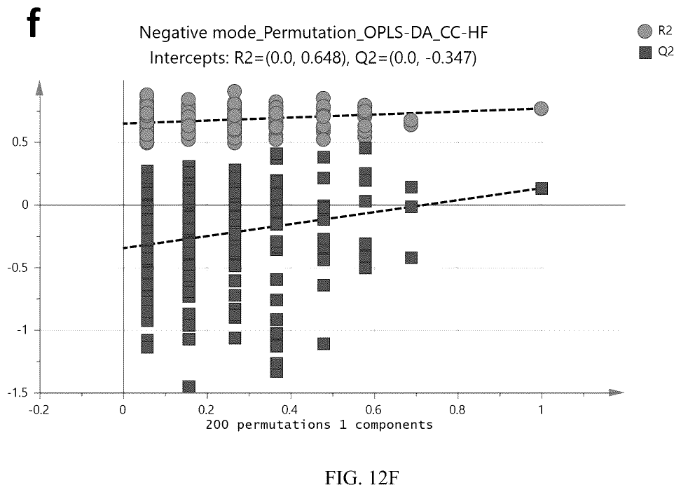

Based on the analysis on features of the positive mode, the LF group was distinctly separated from clusters of the HF and CC groups in the unsupervised PCA scores plot, suggesting that the low-fat diet distinguished serum metabolic characterization of the LF group from the metabolites of the high-fat diet groups (the HF and CC groups). In addition, the CC group was largely separated from the HF group by component 3 in the PCA scores plot (FIG. 12A). In the supervised OPLS-DA plot (FIG. 12B), the HF and CC groups were significantly separated into two clusters that were far away from each other. The model presented an R²X_{(cum)} at 0.566, a goodness-of-fit R² at 0.956, and a goodness-of prediction Q² at 0.781. Permutation test was further performed to validate the OPLS-DA model (FIG. 12C). As a result, all permutated blue Q²-values to the left were lower than the original point to the right (Axis Y=0.781), and the blue regression line of Q²-points intersected the left vertical axis below zero (−0.574), suggesting that the OPLS-DA separation was valid without overfitting, see Jing L, et al., (2019) Sci Rep 9:1-10.

Features of the negative mode did not exhibit good separation between the low-fat diet group and the high-fat diet groups. However, a trend of separation was observed between the CC and HF groups (FIG. 12D). The OPLS-DA model of the negative mode showed a significant group classification (FIG. 12E), and presented an R²X_{(cum)} at 0.490, a goodness-of-fit R² at 0.768, and a goodness-of prediction Q² at 0.131. Although some permutated blue Q²-values were higher than the original point (Axis Y=0.131), the OPLS-DA model did not overfit the data considering that the blue regression line of Q²-points intersected the vertical axis below zero (−0.347) (FIG. 12F).

With the threshold VIP values≥2 and P<0.05, 65 discriminatory features were obtained from OPLS-DA analysis. Of which, 51 were generated from the data of the positive ionization mode and 14 from the negative mode. The enrichment analysis classified the 53 structurally identified metabolites into categories of amino acids and peptides (9), acylcarnitines (7), glycerophosphocholines (5), benzamides (4), hydroxy acids (2), pyrimidines (2), indoles (1), carboxylic acids (1), pyrrolines (1), organic carbonic acids (1), TCA acids (1), fatty amides (1), and others. Nine of these identified metabolites were up-regulated by the C. cochlearium treatment while the others were down-regulated. Among them, 24 identified biomarkers hit 24 pathways of mouse metabolism, see Table 4.

TABLE 4
Altered biomarkers and pathways by C. cochlearium treatment

| | Match | | |
|---|---|---|---|
| Pathway Name | Status | Metabolite hit | Regulation |
| Aminoacyl-tRNA biosynthesis | 6/48 | Glutamate, Glutamine, Arginine, | Down |
| | | Asparagine, Proline, Phenylalanine | or UP ^{a} |
| Arginine and proline | 6/38 | Glutamate, Arginine, Proline, | Down |
| metabolism | | Ornithine, S-Adenosyl-L- | or UP ^{a} |
| | | methionine (AcCa), 1-Pyrroline-4- | |
| | | hydroxy-2-carboxylate | |
| Arginine biosynthesis | 4/14 | Glutamate, Glutamine, Arginine, | Down |
| | | Ornithine | or UP ^{a} |
| Alanine, aspartate and glutamate | 3/28 | Glutamate, Glutamine, Asparagine | Down |
| metabolism | | | |
| Glutathione metabolism | 3/28 | Glutamate, Ornithine, 5-Oxoproline | Down |
| | | (Pyroglutamic acid) | |
| Glyoxylate and dicarboxylate | 3/32 | Glutamate, Glutamine, cis- | Down |
| metabolism | | Aconitate | |
| D-Glutamine and D-glutamate | 2/6 | Glutamate, Glutamine | Down |
| metabolism | | | |
| Nitrogen metabolism | 2/6 | Glutamate, Glutamine | Down |
| Butanoate metabolism | 2/15 | Glutamate, (R)-3-Hydroxybutanoate | Down |
| Linoleic acid metabolism | 1/5 | Glutamate | Down |
| Histidine metabolism | 1/16 | Glutamate | Down |
| Porphyrin and chlorophyll | 1/30 | Glutamate | Down |
| metabolism | | | |
| Purine metabolism | 1/66 | Glutamine | Down |
| Pyrimidine metabolism | 2/39 | Glutamine, 5,6-Dihydrouracil | Down |
| Pantothenate and CoA | 1/19 | 5,6-Dihydrouracil | Down |
| biosynthesis | | | |
| beta-Alanine metabolism | 1/21 | 5,6-Dihydrouracil | Down |
| Phenylalanine, tyrosine and | 1/4 | Phenylalanine | Down |
| tryptophan biosynthesis | | | |
| Phenylalanine metabolism | 1/12 | Phenylalanine | Down |
| Synthesis and degradation of | 1/5 | (R)-3-Hydroxybutanoate | Down |
| ketone bodies | | | |
| Citrate cycle (TCA cycle) | 1/20 | cis-Aconitate | Down |
| Mitochondrial L-carnitine | — | Acylcarnitine (AcCa) | Down |
| shuttle | | | |
| Glycerophospholipid | 2/36 | 1-Acyl-sn-glycero-3- | Down |
| metabolism | | phosphocholine (LPC), | or UP ^{a} |
| | | Phosphatidylcholine (PC) | |
| alpha-Linolenic acid metabolism | 1/13 | Phosphatidylcholine (PC) | Down |
| Arachidonic acid metabolism | 1/36 | Phosphatidylcholine (PC) | Down |

^{a} Pathways are up-regulated in terms of arginine, LPC(18:2) or LPC(22:6)

Correspondingly, most of the matched pathways were down-regulated, except for those related to arginine, LPC(18:2) and LPC(22:6).

### Correlations Between Probiotic Effects and Serum Biomarkers

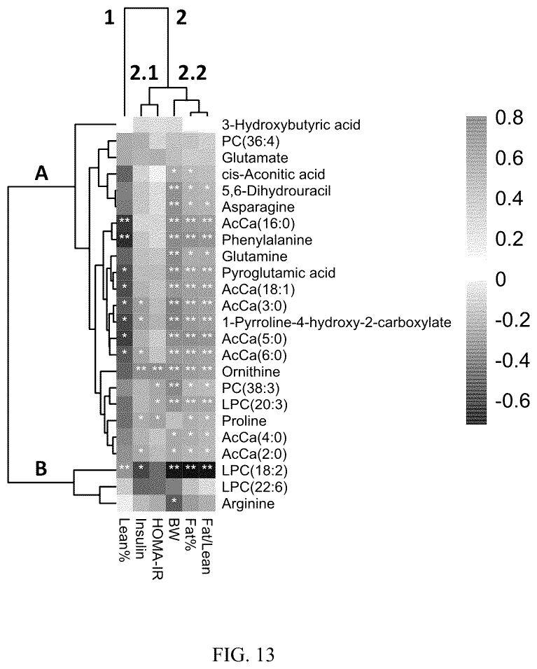

The heat map (FIG. 13) summarized the 24 pathway-matched biomarkers into two major clusters, which differentiated the 21 down-regulated biomarkers (cluster A) from the 3 up-regulated ones (cluster B). The six phenotype variables were also separated into two primary clusters, with body lean percentage (cluster 1) as an opposite characterization to the others (cluster 2). The insulin related variables (cluster 2.1) were further clustered from the body mass related variables (cluster 2.2).

In this example, the probiotic effects of C. cochlearium supplementation on DIO mice, and mechanisms of actions were assessed via chemical characterization. C. cochlearium administration significantly reduced body weight gains and body fat composition on mice. C. cochlearium intake also significantly improved insulin sensitivity and reduced blood glucose levels on mice.

Body weight change is a result of energy balance between absorption and expenditure. The anti-obesity effect of C. cochlearium is likely attributed to increased energy expenditure, given that there was no difference on the net calorie absorption between the HF and CC groups. Diet-induced energy expenditure is different among macronutrients with carbohydrate inducing 2-3 times more energy expenditure than fat does. However, the HF and CC groups were consuming the same high fat diet, so it is believed that 22% lower body weight by C. cochlearium supplementation is achieved through increased other two forms of energy expenditure: resting metabolic rate (RMR) and physical activity. C57BL/6 mice are susceptible to develop obesity on a high-fat diet by lowering fat oxidation. C. cochlearium may stimulate fat oxidation and increase energy expenditure, leading to reduced body weight gain.

This example showed that C. cochlearium produced SCFAs, especially substantial amount of butyrate as compared to other bacteria. These results suggest that C. cochlearium produces butyrate from glutamate.

This example showed that C. cochlearium is a solid butyrate producer and its dietary supplementation reduced obesity development on mice.

C. cochlearium administration significantly altered the serum metabolites on DIO mice. Fifty-three metabolites associated with the beneficial effects of C. cochlearium supplementation were identified. A group of amino acids and peptides accounted for major part (9 out of 53), including glutamate which is associated with obesity and obesity associated insulin resistance in humans. This example showed that C. cochlearium administration decreased serum glutamate, indicating a role of glutamate in mediating anti-obesity effect of C. cochlearium. Table 2 shows that a number of glutamate related biological pathways were also down regulated by C. cochlearium. These pathways include aminoacyl-tRNA biosynthesis (6/48), arginine and proline metabolism (6/38), arginine biosynthesis (4/14), alanine, aspartate and glutamate metabolism (3/28), glutathione metabolism (3/28), and glyoxylate and dicarboxylate metabolism (3/32).

Phenylalanine and ornithine are two other amino acid biomarkers related to obesity or diabetes. In this example, serum levels of phenylalanine and ornithine were positively correlated with mouse body weight, fat mass and fat/lean ratio, or HOMA-IR (P<0.01; FIG. 6), indicating involvement of these two amino acids in the probiotic activity of C. cochlearium.

A correlation test disclosed in this example, revealed that serum asparagine was positively correlated obesity and insulin resistance, while arginine had a negative association (FIG. 13).

The metabolomic analysis applied in this example shows that C. cochlearium treatment significantly altered the serum levels of two phosphatidylcholines (PCs), three lysophosphocholines (LPCs) and seven acylcarnitines (AcCas) at micro- to nano-molar scales.

This example demonstrated that C. cochlearium supplementation significantly reduced serum PC (38:3) and PC (36:4). In this example, serum levels of LPC (22:6) and LPC (18:2) were also lower in the HF group compared with the CC group. However, serum LPC (20:3) in this example exhibited positive correlations with body weight, fat mass, fat/lean ratio, and HOMA-IR (FIG. 13).

Further, seven AcCa species were downregulated in the CC group, and they all were positively associated with body weight, fat mass and fat/lean ratio (FIG. 13). The results suggest that C. cochlearium administration may stimualte the FAO process, subsequently reducing body weight gain and improving insulin sensitivity. It is worth noting that five of the seven AcCa biomarkers identified in this example were short-chain species, and short-chain AcCas were particularly elevated in diabetes subjects.

This example demonstrated that dietary supplementation of C. cochlearium attenuated body weight gain and improved insulin sensitivity in high-fat diet induced obese C57BL/6 mice. Fifty-three discriminatory metabolites were identified between CC and HF groups. Of which, 24 metabolites matched mouse metabolic pathways. Most of these metabolites showed positive correlations with body weight, which included glutamate, phenylalanine, ornithine, PCs, LPCs, AcCas, proline, 5,6-dihydrouracil, pyroglutamic acid, and 1-pyrroline-4-hydroxy-2-carboxylate.

### Items

Item 1. A method of improving one or more of: impaired insulin sensitivity, overweight, obesity, glucose homeostasis dysregulation, and gut microbiota profile dysregulation, comprising: administering an effective amount of isolated C. cochlearium, isolated L. acidophilus or a combination of isolated C. cochlearium and isolated L. acidophilus, formulated together or separately, in a probiotic composition, to a subject in need thereof.

Item 2. The method of item 1, wherein the effective amount of isolated C. cochlearium, isolated L. acidophilus or a combination of isolated C. cochlearium and isolated L. acidophilus, formulated together or separately, is in the range of 10⁵ CFU to 10¹⁵ CFU, 10⁶ CFU to 10¹⁴ CFU, 10⁷ CFU to 10¹³ CFU, 10⁸ CFU to 10¹² CFU, 10⁹ CFU to 10¹¹ CFU, or 10¹⁰ CFU.

Item 3. The method of item 1 or 2, wherein administering the effective amount comprises a single administration of isolated C. cochlearium, isolated L. acidophilus or a combination of isolated C. cochlearium and isolated L. acidophilus.

Item 4. The method of item 1 or 2, wherein administering the effective amount comprises a multiple administrations of isolated C. cochlearium, isolated L. acidophilus or a combination of isolated C. cochlearium and isolated L. acidophilus.

Item 5. The method of item 4, wherein the multiple administrations are performed at regular intervals selected from daily, weekly, monthly, or more frequently, or less frequently.

Item 6. The method of any one of items 1 to 5, wherein the effective amount of isolated C. cochlearium is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

Item 7. The method of any one of items 1 to 6, wherein the effective amount of isolated L. acidophilus is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

Item 8. The method of any one of items 1 to 7, wherein the effective amount of isolated C. cochlearium is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day, in a combination treatment with isolated L. acidophilus in doses of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

Item 9. The method of any one of the preceding items, wherein administering the effective amount is via an enteral route.

Item 10. The method of any one of the preceding items, wherein the subject is human.

Item 11. A probiotic composition comprising: isolated C. cochlearium, isolated L. acidophilus or a combination of isolated C. cochlearium and isolated L. acidophilus for improving one or more of: impaired insulin sensitivity, overweight, obesity, glucose homeostasis dysregulation, and gut microbiota profile dysregulation.

Item 12. The probiotic composition of item 11, further comprising a pharmaceutically acceptable carrier.

Item 13. The probiotic composition of item 11 or 12, formulated as a unit dosage comprising an effective amount of isolated C. cochlearium, isolated L. acidophilus or a combination of isolated C. cochlearium and isolated L. acidophilus.

Item 14. The probiotic composition of item 13, wherein the unit dosage is in the range of 10⁵ CFU to 10¹⁵ CFU, 10⁶ CFU to 10¹⁴ CFU, 10⁷ CFU to 10¹³ CFU, 10⁸ CFU to 10¹² CFU, 10⁹ CFU to 10¹¹ CFU, or 10¹⁰ CFU.

Item 15. A probiotic composition for use in improving one or more of: impaired insulin sensitivity, overweight, obesity, glucose homeostasis dysregulation, and gut microbiota profile dysregulation, comprising: an effective amount of isolated C. cochlearium, isolated L. acidophilus or a combination of isolated C. cochlearium and isolated L. acidophilus, formulated together or separately.

Item 16. The probiotic composition for use according to item 15, wherein the effective amount of isolated C. cochlearium, isolated L. acidophilus or a combination of isolated C. cochlearium and isolated L. acidophilus, formulated together or separately, is in the range of 10⁵ CFU to 10¹⁵ CFU, 10⁶ CFU to 10¹⁴ CFU, 10⁷ CFU to 10¹³ CFU, 10⁸ CFU to 10¹² CFU, 109 CFU to 10¹¹ CFU, or 10¹⁰ CFU.

Item 17. The probiotic composition for use according to item 15 or 16, wherein the effective amount of isolated C. cochlearium is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

Item 18. The probiotic composition for use according to item 15, 16, or 17, wherein the effective amount of isolated L. acidophilus is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

Item 19. The probiotic composition for use according to any one of items 15 to 18, wherein the effective amount of isolated C. cochlearium is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day, in a combination treatment with isolated L. acidophilus in doses of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

Item 20. The probiotic composition for use according to any one of items 15 to 19, further comprising a pharmaceutically acceptable carrier.

Item 21. The probiotic composition for use according to any one of items 15 to 20, formulated as a unit dosage comprising an effective amount of isolated C. cochlearium, isolated L. acidophilus or a combination of isolated C. cochlearium and isolated L. acidophilus.

Item 22. The probiotic composition for use according to any one of items 15 to 21, wherein the unit dosage is in the range of 10⁵ CFU to 10¹⁵ CFU, 10⁶ CFU to 10¹⁴ CFU, 10⁷ CFU to 10¹³ CFU, 10⁸ CFU to 10¹² CFU, 10⁹ CFU to 10¹¹ CFU, or 10¹⁰ CFU.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of improving one or more of: impaired insulin sensitivity, overweight, obesity, glucose homeostasis dysregulation, and gut microbiota profile dysregulation, comprising:
administering an effective amount of isolated C. cochlearium, isolated L. acidophilus or a combination of isolated C. cochlearium and isolated L. acidophilus, formulated together or separately, in a probiotic composition, to a subject in need thereof.

2. The method of claim 1, wherein the effective amount of isolated C. cochlearium, isolated L. acidophilus or a combination of isolated C. cochlearium and isolated L. acidophilus, formulated together or separately, is in the range of 10⁵ CFU to 10¹⁵ CFU, 10⁶ CFU to 10¹⁴ CFU, 10⁷ CFU to 10¹³ CFU, 10⁸ CFU to 10¹² CFU, 10⁹ CFU to 10¹¹ CFU, or 10¹⁰ CFU.

3. The method of claim 1, wherein administering the effective amount comprises a single administration of isolated C. cochlearium, isolated L. acidophilus or a combination of isolated C. cochlearium and isolated L. acidophilus.

4. The method of claim 1, wherein administering the effective amount comprises a multiple administrations of isolated C. cochlearium, isolated L. acidophilus or a combination of isolated C. cochlearium and isolated L. acidophilus.

5. The method of claim 4, wherein the multiple administrations are performed at regular intervals selected from daily, weekly, monthly, or more frequently, or less frequently.

6. The method of claim 1, wherein the effective amount of isolated C. cochlearium is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

7. The method of claim 1, wherein the effective amount of isolated L. acidophilus is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10² CFU/day, 10⁹ CFU/day to 10¹¹ CFU/day, or 10¹⁰ CFU/day.

8. The method of claim 1, wherein the effective amount of isolated C. cochlearium is in the range of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day, in a combination treatment with isolated L. acidophilus in doses of 10⁵ CFU/day to 10¹⁵ CFU/day, 10⁶ CFU/day to 10¹⁴ CFU/day, 10⁷ CFU/day to 10¹³ CFU/day, 10⁸ CFU/day to 10¹² CFU/day, 10⁹ CFU/day to 10¹¹ CFU/day, or 10¹⁰ CFU/day.

9. The method of claim 1, wherein administering the effective amount is via an enteral route.

10. The method of claim 1, wherein the subject is human.

11. A probiotic composition comprising: isolated C. cochlearium, isolated L. acidophilus or a combination of isolated C. cochlearium and isolated L. acidophilus for improving one or more of: impaired insulin sensitivity, overweight, obesity, glucose homeostasis dysregulation, and gut microbiota profile dysregulation.

12. The probiotic composition of claim 11, further comprising a pharmaceutically acceptable carrier.

13. The probiotic composition of claim 11, formulated as a unit dosage comprising an effective amount of isolated C. cochlearium, isolated L. acidophilus or a combination of isolated C. cochlearium and isolated L. acidophilus.

14. The probiotic composition of claim 13, wherein the unit dosage is in the range of 10⁵ CFU to 10¹⁵ CFU, 10⁶ CFU to 10¹⁴ CFU, 10⁷ CFU to 10¹³ CFU, 10⁸ CFU to 10¹² CFU, 109 CFU to 10¹¹ CFU, or 10¹⁰ CFU.
